SEQUENCE LISTING (129 sequences)

SEQ ID NO: 1 - DNA, 20 bp, Artificial Sequence
  Synthetic 1
caattacacagtacgtgtct

SEQ ID NO: 2 - DNA, 20 bp, Artificial Sequence
  Synthetic 2
ctggagaacagaattgcaga

SEQ ID NO: 3 - DNA, 20 bp, Artificial Sequence
  Synthetic 3
tagcaatcatattcacgggc

SEQ ID NO: 4 - DNA, 20 bp, Artificial Sequence
  Synthetic 4
atggtcattcagcatggact

SEQ ID NO: 5 - DNA, 20 bp, Artificial Sequence
  Synthetic 5
gaggccagtacagaaaacat

SEQ ID NO: 6 - DNA, 20 bp, Artificial Sequence
  Synthetic 6
tggaaaagtatccctgagga

SEQ ID NO: 7 - DNA, 20 bp, Artificial Sequence
  Synthetic 7
tgctagtctggagttgatca

SEQ ID NO: 8 - DNA, 20 bp, Artificial Sequence
  Synthetic 8
tggcttcctgctaaacagta

SEQ ID NO: 9 - DNA, 20 bp, Artificial Sequence
  Synthetic 9
tggcttcctgctaaacagta

SEQ ID NO: 10 - DNA, 20 bp, Artificial Sequence
  Synthetic 10
gacgtctgtctacattgaat

SEQ ID NO: 11 - DNA, 20 bp, Artificial Sequence
  Synthetic 11
ttctgaagataccgttaata

SEQ ID NO: 12 - DNA, 20 bp, Artificial Sequence
  Synthetic 12
ttctgaagataccgttaata

SEQ ID NO: 13 - DNA, 20 bp, Artificial Sequence
  Synthetic 13
atccaaactgatttcatccc

SEQ ID NO: 14 - DNA, 20 bp, Artificial Sequence
  Synthetic 14
gttctcatgctgtaatgagc

SEQ ID NO: 15 - DNA, 20 bp, Artificial Sequence
  Synthetic 15
tgtattggacgttctaaatg

SEQ ID NO: 16 - DNA, 20 bp, Artificial Sequence
  Synthetic 16
tgggaaaacctatcggaaga

SEQ ID NO: 17 - DNA, 20 bp, Artificial Sequence
  Synthetic 17
aaagtggtggtatacgatat

SEQ ID NO: 18 - DNA, 20 bp, Artificial Sequence
  Synthetic 18
gaatgctatgcttagattag

SEQ ID NO: 19 - DNA, 20 bp, Artificial Sequence
  Synthetic 19
ccctttcacccatacacatt

SEQ ID NO: 20 - DNA, 20 bp, Artificial Sequence
  Synthetic 20
agacagacactcggtagcaa

SEQ ID NO: 21 - DNA, 20 bp, Artificial Sequence
  Synthetic 21
taaccaggtaatattggcaa

SEQ ID NO: 22 - DNA, 20 bp, Artificial Sequence
  Synthetic 22
acacacgctttttacctgag

SEQ ID NO: 23 - DNA, 20 bp, Artificial Sequence
  Synthetic 23
accacctatcatctaatgat

SEQ ID NO: 24 - DNA, 20 bp, Artificial Sequence
  Synthetic 24
accacctatcatctaatgat

SEQ ID NO: 25 - DNA, 20 bp, Artificial Sequence
  Synthetic 25
gccaacacgagctgactctg

SEQ ID NO: 26 - DNA, 20 bp, Artificial Sequence
  Synthetic 26
ttggggaactttcaatgcag

SEQ ID NO: 27 - DNA, 20 bp, Artificial Sequence
  >Synthetic 27
ttggggaactttcaatgcag

SEQ ID NO: 28 - DNA, 20 bp, Artificial Sequence
  Synthetic 28
gaaaccaaacacaacccatc

SEQ ID NO: 29 - DNA, 20 bp, Artificial Sequence
  Synthetic 29
aaagcgagcaagagaatccc

SEQ ID NO: 30 - DNA, 20 bp, Artificial Sequence
  Synthetic 30
aaagcgagcaagagaatccc

SEQ ID NO: 31 - DNA, 20 bp, Artificial Sequence
  Synthetic 31
acctgtgggcatgttggtga

SEQ ID NO: 32 - DNA, 20 bp, Artificial Sequence
  Synthetic 32
ctggctgcacaaccacaatt

SEQ ID NO: 33 - DNA, 20 bp, Artificial Sequence
  Synthetic 33
cttacagcagtagtatcatg

SEQ ID NO: 34 - DNA, 20 bp, Artificial Sequence
  Synthetic 34
tgattctctgtcatgcctgc

SEQ ID NO: 35 - DNA, 20 bp, Artificial Sequence
  Synthetic 35
atggttttatatggagacac

SEQ ID NO: 36 - DNA, 20 bp, Artificial Sequence
  Synthetic 36
tgtctacctgaccaatcgat

SEQ ID NO: 37 - DNA, 20 bp, Artificial Sequence
  Synthetic 37
aacctgccataattttcgtt

SEQ ID NO: 38 - DNA, 20 bp, Artificial Sequence
  Synthetic 38
agatgggtggtatgctgtta

SEQ ID NO: 39 - DNA, 20 bp, Artificial Sequence
  Synthetic 39
tcaaagagcataccctatac

SEQ ID NO: 40 - DNA, 20 bp, Artificial Sequence
  Synthetic 40
agtttcggcactcaagcgcc

SEQ ID NO: 41 - DNA, 20 bp, Artificial Sequence
  Synthetic 41
tgtaggaaatcatcataatc

SEQ ID NO: 42 - DNA, 20 bp, Artificial Sequence
  Synthetic 42
cacctggccatcgatcatgt

SEQ ID NO: 43 - DNA, 20 bp, Artificial Sequence
  Synthetic 43
catcatggtcacctacaacg

SEQ ID NO: 44 - DNA, 20 bp, Artificial Sequence
  Synthetic 44
gggcagtcataatctcaagg

SEQ ID NO: 45 - DNA, 20 bp, Artificial Sequence
  Synthetic 45
gaatacgtggccagagtctg

SEQ ID NO: 46 - DNA, 20 bp, Artificial Sequence
  Synthetic 46
ccatcggatccagcaccagc

SEQ ID NO: 47 - DNA, 20 bp, Artificial Sequence
  Synthetic 47
agcggtagaagtgcatcacc

SEQ ID NO: 48 - DNA, 92 bp, Artificial Sequence
  Synthetic 48
gcaaaatatgtggtcacactttgtggagacaggttctttgttcaactccagactagcagggtagggggggagaaaaagaaaataaatgaggc

SEQ ID NO: 49 - DNA, 92 bp, Artificial Sequence
  Synthetic 49
gcaaaatatgtggtcacactttgtggagacaggttctttaatcaactccagactagcagggtagggggggagaaaaagaaaataaatgaggc

SEQ ID NO: 50 - DNA, 88 bp, Artificial Sequence
  Synthetic 50
ccaaggttagagagttggacactgagactggttgcctgctaaacagtatgataaagaacagtcaagcaattgttggccagttctgtgc

SEQ ID NO: 51 - DNA, 88 bp, Artificial Sequence
  Synthetic 51
ccaaggttagagagttggacactgagactcgtttcctgctaaacagtatgataaagaacagtcaagcaattgttggccagttctgtgc

SEQ ID NO: 52 - DNA, 88 bp, Artificial Sequence
  Synthetic 52
ccaaggttagagagttggacactgagactggcttcctgctaaacagtatgataaagaacagtcaagcaattgttggccagttctgtgc

SEQ ID NO: 53 - DNA, 88 bp, Artificial Sequence; identical to SEQ ID NO: 51
  Synthetic 53

SEQ ID NO: 54 - DNA, 84 bp, Artificial Sequence
  Synthetic 54
gcaattattattaaatacttaaaaaacctgagacccttacctaattcaatgcagacagacgtcttttgaggttgtatccgctgc

SEQ ID NO: 55 - DNA, 84 bp, Artificial Sequence
  Synthetic 55
gcaattattattaaatacttaaaaaacctgagacccttacctaattcaatatagacagacgtcttttgaggttgtatccgctgc

SEQ ID NO: 56 - DNA, 80 bp, Artificial Sequence
  Synthetic 56
ggttctctttgactcacctgcaataagttgctttattatcggtatcttcagaagaatcagatcctaaaaaatttcccccc

SEQ ID NO: 57 - DNA, 80 bp, Artificial Sequence
  Synthetic 57
ggttctctttgactcacctgcaataagttgctttatttacggtatcttcagaagaatcagatcctaaaaaatttcccccc

SEQ ID NO: 58 - DNA, 81 bp, Artificial Sequence
  Synthetic 58
ccagcttcatagacaaaggttctctttgactcacctacaataagttgctttattaacggtatcttcagaagaatcagatcc

SEQ ID NO: 59 - DNA, 81 bp, Artificial Sequence
  Synthetic 59
ccagcttcatagacaaaggttctctttgactcacctgcagtaagttgctttattaacggtatcttcagaagaatcagatcc

SEQ ID NO: 60 - DNA, 82 bp, Artificial Sequence
  Synthetic 60
gccattacccttttttgcagaatccaaactgatttcatccctcgttccctgaggggtgatttgtaacaattcttgatctccc

SEQ ID NO: 61 - DNA, 82 bp, Artificial Sequence
  Synthetic 61
gccattacccttttttgcagaatccaaactgatttcatccctcgtaccttgaggggtgatttgtaacaattcttgatctccc

SEQ ID NO: 62 - DNA, 86 bp, Artificial Sequence
  Synthetic 62
ctacattcattctgtctttagtgagtcataaactgctgttctcatgctgtaatgagcttgcatgagtatttgtgccacatggctcc

SEQ ID NO: 63 - DNA, 86 bp, Artificial Sequence
  Synthetic 63
ctacattcattctgtctttagtgagcaataaactgctgttctcatgctgtaatgagcttgcatgagtatttgtgccacatggctcc

SEQ ID NO: 64 - DNA, 85 bp, Artificial Sequence
  Synthetic 64
cactggccagtaagtctattttctctgaagaaccagactattcatctacttcatttagaacgtccaatacatcagctactttggc

SEQ ID NO: 65 - DNA, 85 bp, Artificial Sequence
  Synthetic 65
cactggccagtaagtctattttctctgaagaaccagagtattcatctacttcatttagaacgtccaatacatcagctactttggc

SEQ ID NO: 66 - DNA, 84 bp, Artificial Sequence
  Synthetic 66
cagttacatggcttaagttggggaggcttgctttcttccgataggttttcctaaatattttgtcttcaatattactctctactg

SEQ ID NO: 67 - DNA, 84 bp, Artificial Sequence
  Synthetic 67
cagttacatggcttaagttggggaggcttgctttcttccgataggtttttccaaatattttgtcttcaatattactctctactg

SEQ ID NO: 68 - DNA, 84 bp, Artificial Sequence
  Synthetic 68
cttacatttagttttaacaaatgacttgatgggaaaaagtggtggtatacaatatggattttgtaaaagtccatgtttatttgg

SEQ ID NO: 69 - DNA, 84 bp, Artificial Sequence
  Synthetic 69
cttacatttagttttaacaaatgacttgatgggaaaaagtggtggtatgcgatatggattttgtaaaagtccatgtttatttgg

SEQ ID NO: 70 - DNA, 89 bp, Artificial Sequence
  Synthetic 70
ggaagactttgtttatagacctcaggttgcaaaactcctaatctaagcatagcattcagttttggccctctgtttctacctagttctgc

SEQ ID NO: 71 - DNA, 89 bp, Artificial Sequence
  Synthetic 71
ggaagactttgtttatagacctcaggttgcaaaacacctaatctaagcatagcgttcaattttggccctctgtttctacctagttctgc

SEQ ID NO: 72 - DNA, 84 bp, Artificial Sequence
  Synthetic 72
ggactctaatttcttggcccctcttcggtaactctgagcgaaatgtgtatgggtgaaagggctaggactcctgctaagctctcc

SEQ ID NO: 73 - DNA, 84 bp, Artificial Sequence
  Synthetic 73
ggactctaatttcttggcccctcttcggtatccctgagcgaaatgtgtatgggtgaaagggctaggactcctgctaagctctcc

SEQ ID NO: 74 - DNA, 87 bp, Artificial Sequence
  Synthetic 74
gctattcttcaatgataataaattctcctctgtgttcttagacagacacttggtagcaacagtgctatgcctagtagactgagaagg

SEQ ID NO: 75 - DNA, 87 bp, Artificial Sequence
  Synthetic 75
gctattcttcaatgataataaattctcctctgtgttcttagacagacattcggtagcaacagtgctatgcctagtagactgagaagg

SEQ ID NO: 76 - DNA, 92 bp, Artificial Sequence
  Synthetic 76
cctcactaaggtgatgttcctgagatgcttttgccaatactacctggttactgcagtcatttaagctattcttcaatgataataaattctcc

SEQ ID NO: 77 - DNA, 92 bp, Artificial Sequence
  Synthetic 77
cctcactaaggtgatgttcctgagatgcttttgccaagattacctggttactgcagtcatttaagctattcttcaatgataataaattctcc

SEQ ID NO: 78 - DNA, 85 bp, Artificial Sequence
  Synthetic 78
gcaaaggacaccacacacacgcatgtgcacacacacacacgctttttacctgaattgttaaaatgtcactctgagaggatagccc

SEQ ID NO: 79 - DNA, 85 bp, Artificial Sequence
  Synthetic 79
gcaaaggacaccacacacacgcatgtgcacacacacacacgctttttacctgagttgttaagatgtcactctgagaggatagccc

SEQ ID NO: 80 - DNA, 83 bp, Artificial Sequence
  Synthetic 80
ctcccagagcaactgtgcatgtaccacctattatctaatgatggacatttagaaggggatgacctagaaagataaatggaagg

SEQ ID NO: 81 - DNA, 83 bp, Artificial Sequence
  Synthetic 81
ctcccagagcaactgtgcatgtaccacctgtcatctaatgatggacatttagaaggggatgacctagaaagataaatggaagg

SEQ ID NO: 82 - DNA, 83 bp, Artificial Sequence
  Synthetic 82
ctcccagagcaactgtgcatgtaccacctctcatctaatgatggacatttagaaggggatgacctagaaagataaatggaagg

SEQ ID NO: 83 - DNA, 83 bp, Artificial Sequence; identical to SEQ ID NO: 81
  Synthetic 83

SEQ ID NO: 84 - DNA, 84 bp, Artificial Sequence
  Synthetic 84
gcagaggttgaagatggtatgttgccaacacgagctgactatggagctctgtcttcagaaggatcagattcagggtcatcagag

SEQ ID NO: 85 - DNA, 84 bp, Artificial Sequence
  Synthetic 85
gcagaggttgaagatggtatgttgccaacacgagctgattctggagctctgtcttcagaaggatcagattcagggtcatcagag

SEQ ID NO: 86 - DNA, 101 bp, Artificial Sequence
  Synthetic 86
gcagcagctggactctgggcagattctgcaactttcaattagggaactttcaatgcagacgttgaagatggtatgttgccaacacgagctgactctggggc

SEQ ID NO: 87 - DNA, 101 bp, Artificial Sequence
  Synthetic 87
gcagcagctggactctgggcagattctgcaactttcaactggggaactttcaatgcagacgttgaagatggtatgttgccaacacgagctgactctggggc

SEQ ID NO: 88 - DNA, 101 bp, Artificial Sequence; identical to SEQ ID NO: 87
  Synthetic 88

SEQ ID NO: 89 - DNA, 101 bp, Artificial Sequence
  Synthetic 89
gcagcagctggactctgggcagattctgcaactttcaattgtggaactttcaatgcagacgttgaagatggtatgttgccaacacgagctgactctggggc

SEQ ID NO: 90 - DNA, 93 bp, Artificial Sequence
  Synthetic 90
ccattgaccacatctcctctgacttcaaaatcatgccgaaagaaaccaaacacaacccatcacgataagagaaagagaagcttccttcaatgg

SEQ ID NO: 91 - DNA, 93 bp, Artificial Sequence
  Synthetic 91
ccattgaccacatctcctctgacttcaaaatcatgctgaaagaacccaaacacaacccatcacgataagagaaagagaagcttccttcaatgg

SEQ ID NO: 92 - DNA, 83 bp, Artificial Sequence
  Synthetic 92
gagggagggagctttacctttctgtcttgggattctcttggtcgctttggaccttggtggtttcttccattgaccacatctcc

SEQ ID NO: 93 - DNA, 83 bp, Artificial Sequence
  Synthetic 93
gagggagggagctttacctttctgtcttgggattctctagctcgctttggaccttggtggtttcttccattgaccacatctcc

SEQ ID NO: 94 - DNA, 83 bp, Artificial Sequence
  Synthetic 94
gagggagggagctttacctttctgtcttgggattctcttgttcgctttggaccttggtggtttcttccattgaccacatctcc

SEQ ID NO: 95 - DNA, 83 bp, Artificial Sequence; identical to SEQ ID NO: 93
  Synthetic 95

SEQ ID NO: 96 - DNA, 88 bp, Artificial Sequence
  Synthetic 96
ggaactctggggttctcccaggctcttacctgtgggcatgttggtgaacggcacatagcaacagatttctagccccctgaagatctgg

SEQ ID NO: 97 - DNA, 88 bp, Artificial Sequence
  Synthetic 97
ggaactctggggttctcccaggctcttacctgtgggcatgttggtgaacggaccatagcaacagatttctagccccctgaagatctgg

SEQ ID NO: 98 - DNA, 86 bp, Artificial Sequence
  Synthetic 98
ggaagccattgtcctctgtccaggcatctgcctgcacaaccacaattggatggacaccctggatccccaggaaggaaagagcattc

SEQ ID NO: 99 - DNA, 86 bp, Artificial Sequence
  Synthetic 99
ggaagccattgtcctctgtccaggcatccggctgcacaaccacaattggatggacaccctggatccccaggaaggaaagagcattc

SEQ ID NO: 100 - DNA, 84 bp, Artificial Sequence
  Synthetic 100
cagtcagaaatgaagaagcatctaaaactgtatttcctcacgatactactgctgtaagtaaatatgacattgattagactgttg

SEQ ID NO: 101 - DNA, 84 bp, Artificial Sequence
  Synthetic 101
cagtcagaaatgaagaagcatctgagactgtatttcctcacgatactactgctgtaagtaaatatgacattgattagactgttg

SEQ ID NO: 102 - DNA, 86 bp, Artificial Sequence
  Synthetic 102
gttatttattaccccagaagctgattctctgtcatgcctgcaagaaggacagtgtgaaaaggatccaaaaagcaaaaaagtttcag

SEQ ID NO: 103 - DNA, 86 bp, Artificial Sequence
  Synthetic 103
gttatttattaccccagaagctgattctctgtcatgcctgcaagaaggacagtgtgaaaacgatccaaaaagcaaaaaagtttcag

SEQ ID NO: 104 - DNA, 89 bp, Artificial Sequence
  Synthetic 104
cgaacccattttcaagaactctaccatggttttatatggagatacaggtaataaacaagcaacccaagtgtcaattaaaaaagatttgg

SEQ ID NO: 105 - DNA, 89 bp, Artificial Sequence
  Synthetic 105
cgaacccattttcaagaactctaccatggttttatatggagatacaggtgacaaacaagcaacccaagtgtcaattaaaaaagatttgg

SEQ ID NO: 106 - DNA, 87 bp, Artificial Sequence
  Synthetic 106
gctgatcttcatgtcataatgaatgctccatcgattggtcaggtagacagcagcaggcaatttgaaggtacagttgaaattaaacgg

SEQ ID NO: 107 - DNA, 87 bp, Artificial Sequence
  Synthetic 107
gctgatcttcatgtcataatgaatgctccatcgattggtcaggtagacagcagcaaacaatttgaaggtacagttgaaattaaacgg

SEQ ID NO: 108 - DNA, 83 bp, Artificial Sequence
  Synthetic 108
cagtaaagtaattaaggaaaacaacgagaataaataaaaaatttgtcaaacgaaaattatggcaggttgttacgaggcattgg

SEQ ID NO: 109 - DNA, 83 bp, Artificial Sequence
  Synthetic 109
cagtaaagtaattaaggaaaacaacgagaataaatccaaaatttgtcaaacgaaaattatggcaggttgttacgaggcattgg

SEQ ID NO: 110 - DNA, 84 bp, Artificial Sequence
  Synthetic 110
gatacccaaaaagtggccattattgaacttacagatgggtggtatgctattaaagcccagttagatcctcccctcttagctgtc

SEQ ID NO: 111 - DNA, 84 bp, Artificial Sequence
  Synthetic 111
gatacccaaaaagtggccattattgaacttacagatgggtggtatgctgtaaaagcccagttagatcctcccctcttagctgtc

SEQ ID NO: 112 - DNA, 85 bp, Artificial Sequence
  Synthetic 112
ggaaatgttggttgtgttgatgtaattattcaaagagcataccctatccgggtatgatgtattcttgaaacttaccatatatttc

SEQ ID NO: 113 - DNA, 85 bp, Artificial Sequence
  Synthetic 113
ggaaatgttggttgtgttgatgtaattattcaaagagcatatcctatccaggtatgatgtattcttgaaacttaccatatatttc

SEQ ID NO: 114 - DNA, 87 bp, Artificial Sequence
  Synthetic 114
cccttctttcactcagggatgatggcgccacttcctcagtttcgtcactcaagcgcctcgaacggagtcagtggacggataagatgg

SEQ ID NO: 115 - DNA, 87 bp, Artificial Sequence
  Synthetic 115
cccttctttcactcagggatgatggcgccacttcctcagtttcggccctcaagcgcctcgaacggagtcagtggacggataagatgg

SEQ ID NO: 116 - DNA, 88 bp, Artificial Sequence
  Synthetic 116
gacattgagacgaccaaactgcccctcaagttttctgatgctgagacagatcagattatgatgatttcctacatgatcgatggccagg

SEQ ID NO: 117 - DNA, 88 bp, Artificial Sequence
  Synthetic 117
gacattgagacgaccaaactgcccctcaagtttcctgatgccgagacagatcagattatgatgatttcctacatgatcgatggccagg

SEQ ID NO: 118 - DNA, 83 bp, Artificial Sequence
  Synthetic 118
gctgagacagaccagattatgatgatttcgtacatgatcggtggccaggtgagcaggtggcttctgggaagtaagctcctggg

SEQ ID NO: 119 - DNA, 83 bp, Artificial Sequence
  Synthetic 119
gctgagacagaccagattatgatgatttcgtacatgattgatggccaggtgagcaggtggcttctgggaagtaagctcctggg

SEQ ID NO: 120 - DNA, 83 bp, Artificial Sequence
  Synthetic 120
ggtttgaacacgtccaggagaccaaacccaccatcatggtcacctacgacggagacttttttgactggtgagtctgtgtcttc

SEQ ID NO: 121 - DNA, 83 bp, Artificial Sequence
  Synthetic 121
ggtttgaacacgtccaggagaccaaacccaccatcatggtcacctacaatggagacttttttgactggtgagtctgtgtcttc

SEQ ID NO: 122 - DNA, 84 bp, Artificial Sequence
  Synthetic 122
ggtgggtgaagagggacagttaccttcctgtgggcagtcataatgtcaaggccgccgccaaggccaagctaggctatgatcccg

SEQ ID NO: 123 - DNA, 84 bp, Artificial Sequence
  Synthetic 123
ggtgggtgaagagggacagttaccttcctgtgggcagtcataatctgaaggccgccgccaaggccaagctaggctatgatcccg

SEQ ID NO: 124 - DNA, 83 bp, Artificial Sequence
  Synthetic 124
gatggccctgctctctggcgttctcttctcagactctgcccacgtattctgtgtcagatgctgtcgccacttactacctgtac

SEQ ID NO: 125 - DNA, 83 bp, Artificial Sequence
  Synthetic 125
gatggccctgctctctggcgttctcttctcagactctggccacatattctgtgtcagatgctgtcgccacttactacctgtac

SEQ ID NO: 126 - DNA, 86 bp, Artificial Sequence
  Synthetic 126
cagccagtcgcagcgaataccatcggatccagcaccagctcgagtcagagaagatcccccccttgttcccagaggggccagctcgg

SEQ ID NO: 127 - DNA, 86 bp, Artificial Sequence
  Synthetic 127
cagccagtcgcagcgaataccatcggatccagcaccagctcgagtcagagaagtttccccccttgttcccagaggggccagctcgg

SEQ ID NO: 128 - DNA, 85 bp, Artificial Sequence
  Synthetic 128
cccagtaccacaacatctatgcagacaatcaggtgatgcacttctaccgctggctttggtcgccatcctctctgcttcatgaccc

SEQ ID NO: 129 - DNA, 85 bp, Artificial Sequence
  Synthetic 129
cccagtaccacaacatctatgcagacaatcaggtgatgcacttctaccgctggcttcgttcgccatcctctctgcttcatgaccc

(19) United States
(12) Patent Application Publication
(10) Pub. No.: US 2023/0227818 A1
(43) Pub. Date: Jul. 20, 2023

(54) CLINICALLY APPLICABLE CHARACTERIZATION OF GENETIC VARIANTS BY GENOME EDITING

(71) Applicants: INSTITUT NATIONAL DE LA SANTE ET DE LA RECHERCHE MEDICALE (INSERM), Paris (FR); UNIVERSITE D'ANGERS, Angers Cedex 01 (FR); ICO (INSTITUT DE CANCÉROLOGIE DE L'OUEST), Angers Cedex 02 (FR)

(72) Inventors: Amandine BILLAUD, Angers (FR); Louise-Marie CHEVALIER, Angers (FR); Alain MOREL, Angers (FR)

(21) Appl. No.: 17/998,442

(22) PCT Filed: May 12, 2021
(86) PCT No.: PCT/EP2021/062697

§ 371 Date: Nov. 10, 2022

(30) Foreign Application Priority Data
May 12, 2020 (EP) 20305476.2

(51) Int. Cl. C12N 15/11; C12N 15/113; C12Q 1/6827

(52) U.S. Cl. CPC C12N 15/111; C12N 15/1135; C12Q 1/6827; C12N 2310/20; C12N 2320/10

(57) ABSTRACT

The invention relates to the field of personalized medicine, and the ability to administer targeted therapies consequently to biomarkers functional identification. In particular, the invention relates to the field of clinical applicable methods for the characterization, and especially the functional evaluation, of genetic variants in a patient. In particular, the invention relates to the field of the characterization, and classification, of variants of uncertain significance (VUS) or other unreported variants in patients. The in vitro method presented here is effective for the characterization of the functional impact of genetic variants in a patient, in particular of VUS, such as BRCA1 and BRCA2 VUS. The inventors have shown that this experimental framework can be used to obtain the necessary biological evidence of VUS function required for the prescription of targeted treatment within three weeks, which is compatible with use in clinical application.

### FIELD OF THE DISCLOSURE

The invention relates to the field of personalized medicine, and the ability to administer targeted therapies consequently to biomarkers functional identification.

In particular, the invention relates to the field of clinical applicable methods for the characterization, and especially the functional evaluation, of genetic variants in a patient. In particular, the invention relates to the field of the characterization, and classification, of variants of uncertain significance or other unreported variants in patients.

The applications include, but are not limited to, the characterization of genetic variants which are presumed to be involved in the occurrence or re-occurrence of cancers in patients.

### BACKGROUND OF THE DISCLOSURE

Over the last two decades, the therapeutic options available in oncology have evolved towards therapies targeted on the basis of tumoral genetic information. These new treatments improve patient outcomes and have fewer adverse effects. However, the functional significance of many variants of targetable genes remains unknown. In the Clinvar database, 237 934 variants of uncertain significance (VUS) are registered for the total set of genes considered. Most are missense mutations and potential splice site variants. With recent technical improvements and the development of whole-exome and whole-genome sequencing, this number of VUS is likely to rise still further in the coming years, with one in three variants classified as VUS overall, and 80% located on tumor suppressor genes. As VUS affect treatment options and patient management, this trend highlights the need for a method of functional testing, which could be applied in oncology, but also other fields of medicine.

Mutations of the BRCA1 and BRCA2 genes can be used to illustrate this problem. These two tumor suppressor genes have many roles, mostly in genome protection via the homologous recombination (HR) pathway. Inheritable mutations of BRCA1/2 increase the risk of breast cancer (50-80%) and ovarian cancer (40-60%), and have also been implicated in prostatic and pancreatic cancers. With the development of PARP inhibitors, pathogenic mutations of these genes are now biomarkers of response to these treatments. Their multiple interaction domains and protein partners account for the tremendous diversity of variants found in tumors. More than 2660 BRCA1 and 4840 BRCA2 VUS are registered in Clinvar, which includes 35.7% and 45.7% of the total known variants of BRCA1 and BRCA2 respectively. However, databases, such as Clinvar, BRCA exchange and UMD, mostly contain constitutive variants. Somatic variants, which may be detected in only one or two individuals, also exist and many such variants remain unreported. Functional testing in vitro is currently based on transcriptional activation, HR activity and splicing. However, such tests are not necessarily compatible with clinical management in terms of the time taken to obtain results to guide treatment.

CRISPR-Cas9 genome editing has been proposed as a promising tool in the context of discovering gene function for large-scale screens, as reported by Doench. (Am I ready for CRISPR? A user's guide to genetic screens. Nat. Rev. Genet. 19, 67-80 (2017)).

Findlay et al. (Saturation editing of genomic regions by multiplex homology-directed repair. Nature 513, 120-123 (2014)) reports a method coupling CRISPR-Cas9 genome editing with multiplex homology-directed repair (HDR) using a library of donor templates.

US20160076093 and Findlay et al. (Accurate functional classification of thousands of BRCA1 variants with saturation genome editing. Nature 294520 (2018)) also report a method wherein all possible SNVs are simultaneously introduced and concurrently assayed by determining the ratio of the frequency of each SNV to its frequency in a whole plasmid library. However, the above-mentioned variants are meant for high-throughput analysis and their application to the clinical practice is uncertain.

Hence, there remains a need for new tools and methods for meeting the challenges of personalized medicine, and especially of VUS classification.

Thus there remains a need for new tools and methods which are applicable efficiently, reliably, and in a timeframe that is compatible with clinical practice.

More specifically, there remains a need for tools and methods which can be undergone within three weeks.

The invention has for purpose to meet the above-mentioned needs.

### SUMMARY

The invention relates to an in vitro method for characterizing one or more genetic variant(s) of a patient, comprising at least the steps of:

a) bringing into contact a first and a second population of haploid cells with:
a Clustered Regularly Interspaced Short Palindromic Repeats (CRISPR)-CRISPR associated (Cas) (CRISPR-Cas) endonuclease, or an expression system capable of expressing said endonuclease in said first and second population;
a first nucleic acid in conditions suitable for introducing in the first population, after sequence-specific cleavage by the endonuclease, (i) at least one mutation corresponding to the genetic variant(s) of the patient, and (ii) at least one silent, or benign, mutation at the corresponding Protospacer Adjacent Motif (PAM) sequence;
a second nucleic acid in conditions suitable for introducing in the second population, after sequence-specific cleavage by the endonuclease, (i) at least one silent, or benign, mutation at the site of the genetic variant(s), and (ii) at least one silent, or benign, mutation at the corresponding PAM sequence, which is the same as the mutation at the PAM sequence corresponding to the first nucleic acid;

b) culturing said first and second population of haploid cells in a culture medium;

c) determining the occurrence of the genetic variant(s) in the first and second population of haploid cells, thereby characterizing the genetic variant(s) of the patient.

### DESCRIPTION OF THE FIGURES

FIG. 1: Generation of polyclonal LIG4 knock-out HAP1 cells. A. Schematic diagram of the LIG4 DNA region targeted by the gRNA, and localization of the AfIIII restriction site used to knock out this gene in HAP1 cells. B. Schematic diagram of the protocol used to generate the polyclonal LIG4 KO cell line. C. Electrophoresis results following digestion with the AfIIII restriction enzyme for screening of the clones. D. Sanger sequence alignments of the clones selected on basis of AfIIII restriction site loss relative to HAP1 parental cells. E. Sanger electrophoregrams of LIG4 KO clones with insertions or deletions causing frameshifts relative to the HAP1 parental cell line.

FIG. 2: Experimental method for characterizing the functional impact of BRCA1/2 SNVs. A. Experimental process used in this analysis for evaluation of the functional impact of BRCA1/2 mutations. B. Examples of the gRNA and the two corresponding oligonucleotides designed for SNV editing, here for the p.Pro1812Ala mutation of the BRCA1 gene. NGS sequencing results and function scores for this mutation are associated.

FIG. 3: Comparison of editing frequencies between BRCA1/2 variants and silent control SNVs for functional classification. A. Insertion and deletion frequencies determined following the NGS sequencing of BRCA1/2 variants, compared with those for the corresponding silent control. These means include the 8 nt surrounding the PAM sequence. B. Same analysis for the 7 nt surrounding the mutations of interest. C. Analysis of NGS sequencing coverage with the following formula: Mutation of interest (Mutation+Reference control)/Silent control (Mutation+Reference control). The results for benign BRCA1/2 variants are compared with those obtained for pathogenic mutations (Mann-Whitney tests, p=0.8016). D. Determination of the function score following CRIPSR-Cas9 editing and NGS sequencing for benign and pathogenic BRCA1/2 mutations (Mann-Whitney tests, p=0.0079).

FIG. 4: Functional characterization of BRCA1/2 variants of unknown significance. A. Schematic representation of the introns and exons of the BRCA1 gene, showing the location of the variants selected for this analysis. B. Similar representation for the BRCA2 gene. C. Analysis of NGS sequencing coverage with the following formula: Mutation of interest (Mutation+Reference control)/Silent control (Mutation+Reference control). Results for all BRCA1/2 variants characterized by comparison with the classified benign and pathogenic mutations (Mann-Whitney tests, p=0.1934 and p=0.2902). D. Frequencies of insertions and deletions according to the NGS sequencing of BRCA1/2 variants and corresponding silent editing controls and linear regression analysis. These means include the 8 nt surrounding the PAM sequence. Previouslyy characterized variants are shown in colors: benign mutations in green and pathogenic mutations in red. E. Function score evaluation after CRIPSR-Cas9 editing and NGS sequencing of BRCA1/2 variants of unknown significance and comparison with the scores obtained for benign and pathogenic mutations.

FIG. 5: Functional characterization with two distinct KO models and two distinct endonucleases. A. Scores obtained following edition of polyclonal cell lines HAP1 KO XRCC4, by comparison to KO LIG4, of benign or pathogenic mutations of BRCA1. B. Results obtained through NGS sequencing and functional scores corresponding to Cpf1 and Cas9 editing, of the same pathogenic mutation of BRCA1, p.Tyr422X.

### DETAILED DESCRIPTION OF THE DISCLOSURE

Herein, we adapt the concept of saturation genome editing, to the clinical study of genetic variants in a patient. By comparing editing frequencies, by NGS sequencing, between a variant of interest and a silencing mutation classified as benign, we were able to evaluate the functional consequences of 33 mutations of BRCA1/2, including 23 variants of uncertain significance (VUS). We further extend the method to the evaluation of other clinically-relevant variants, including seven variants of POLE, another tumor suppressor gene biomarker for immunotherapy administration, demonstrating the utility of this approach for the characterization of genetic variants within a timeframe compatible with clinical application, and of essential tumor suppressor genes in general. The essentiality of the tested genes, including in a non-limitative manner BRCA1, BRCA2 and POLE genes in the haploid model is also an important feature, making it possible to evaluate function rapidly, within three weeks, compatible with direct clinical application.

The in vitro method presented here is thus effective for the characterization of the functional impact of genetic variants in a patient, in particular of VUS, such as BRCA1 and BRCA2 VUS. More importantly, this experimental framework can be used to obtain the necessary biological evidence of VUS function required for the prescription of targeted treatment within three weeks, which is compatible with use in clinical application.

In particular, the in vitro method is particularly suitable for characterizing genetic variants localized on, or associated to, tumor suppressor genes.

The patient carrying the genomic abnormality therefore benefits from an analysis of his or her own mutation, with potential consequences for relatives. This is particularly important for extremely rare somatic variants, which resemble orphan diseases. The extension of its application to the study of other variants and/or characterization of all essential tumor suppressor genes is hence enabled by the proposed experimental model, including, but not limited to, the field of oncology. At a time at which purely in silico approaches are being used to guide therapeutic decisions, a method evaluating the functional implications of VUS is much needed. The experimental model, and the methods reported herein, will be further developed herebelow and in the examples.

### General Definitions

As used herein, a «Genetic variant», for a given patient, relates to the substitution, the deletion or the insertion of at least one (one or more) nucleotides at a specific position, or genomic region of interest, in the genome of a patient. Non-limiting examples of genetic variants include frameshift, stop gained, start lost, splice acceptor, splice donor, stop lost, inframe indel, missense, splice region, synonymous and copy number variants. Non-limiting types of copy number variants include deletions and duplications. When the substitution consists of a single nucleotide, it may also be referred herein as a single-nucleotide variant (SNV). Types of SNVs which are considered as genetic variants according to the methods of the invention, may thus include SNVs in the non-coding region and SNVs in the coding region. Such genetic variants are generally defined by reference to the sequence most prevalent in a population.

The genetic variants (i.e. variants of uncertain significance or unreported variants) which are particularly considered herein are those which concern essential genes. As used herein, an «essential gene» is a gene for which loss of function results in loss of viability or fitness.

As used herein, the terms «Variant of uncertain significance», or «Variant of uncertain significance», or «VUS», are used interchangeably and refer to a form of genetic variant that has been identified through genetic testing but whose significance to the function of a gene or protein or the health of an organism is not known at the time of characterization.

«Locus of interest» and «genomic region of interest» are used interchangeably herein to mean the region of the genome of the patient.

The terms «at least one» and «one or more» are used interchangeably. Accordingly, the term «at least one» may comprise «two or more», «three or more», «four or more», «five or more», and so on.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Nuclease” and “endonuclease” are used interchangeably herein to mean one or more enzymes or enzyme-containing complexes (which may include protein-nucleic acid complexes such as Cas9 in complex with sgRNAs) which possesses catalytic activity for polynucleotide cleavage, in particular DNA cleavage. Endonucleases which are considered include naturally occurring, non-naturally occurring, recombinant, chimeric and/or heterologous endonucleases, and analogs thereof.

Analogs of endonucleases may include endonucleases which share at least 80% of sequence identity with a given endonuclease, which includes at least 80%; 85%; 90% and 95% of identity, based on an optimum alignment.

The optimum alignment of the sequences for the comparison can be carried out by computer using known algorithms. Entirely preferably, the percentage sequence identity is determined using the CLUSTAL W2 software (version 2.1), the parameters being fixed as «default». By “endonuclease suitable for targeting a genomic region of interest” it is meant any CRISPR/Cas endonuclease, as described above, that is able to target specifically a genomic region of interest of a given cell, and to provide catalytic activity for polynucleotide cleavage on the targeted genomic region.

Thus, said definition may include both:

(i) endonucleases having at least one targeting domain and at least one active domain for polynucleotide cleavage; and/or

(ii) endonuclease having at least one active domain for polynucleotide cleavage, wherein the targeting domain is part of a distinct polypeptide and/or a distinct polynucleotide.

In general, “CRISPR system” or «CRISPR/Cas system» refers collectively to transcripts and other elements involved in the expression of or directing the activity of CRISPR-associated (“Cas”) genes, including sequences encoding one or more of: a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), a single-guide nucleic acid (in particular a single-guide RNA (sgRNA)) or other associated sequences and transcripts from a CRISPR locus needed for targeting a genomic region of interest (i.e. a genetic variant of the patient which is to be characterized).

In some embodiments, one or more elements of a CRISPR system is/are derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides.

Among CRISPR-Cas systems, a type II CRISPR system from Streptococcus pyogenes involves only a single gene encoding the Cas9 protein and two RNAs—a mature CRISPR RNA (crRNA) and a partially complementary trans-acting RNA (tracrRNA)—which are necessary and sufficient for RNA-guided silencing of foreign DNAs.

Accordingly, a CRISPR-Cas system suitable for the methods of the invention may involve a Cas endonuclease and a CRISPR-Cas system guide nucleic acid, such as a CRISPR-Cas system guide RNA that hybridizes with the target sequence.

Examples of CRISPR/Cas endonucleases include class 2 CRISPR/Cas endonucleases, such as: (a) type II CRISPR/Cas proteins, e.g., a Cas9 protein and the like; (b) type IIA CRISPR/Cas proteins, e.g., a Csn2 protein and the like; (c) type IIB CRISPR/Cas proteins, e.g., a Cas4 protein and the like; (d) type IIC CRISPR/Cas proteins; (e) type V CRISPR/Cas proteins, e.g., a Cpf1 polypeptide, a C2c1 polypeptide, a C2c3 polypeptide, and the like; and (f) type VI CRISPR/Cas proteins, e.g., a C2c2 protein, a Cas13b protein, a Cas13c protein, a Cas13d protein and the like.

In particular, “Class 2 CRISPR system” which are considered by the invention include Type II (a sub-type of “class 2”) CRISPR systems such as CRISPR/Cas9 or the more recently characterized CRISPR from Provotella and Francisella 1 (Cpf1) in Zetsche et al. (“Cpf1 is a Single RNA-guided Endonuclease of a Class 2 CRISPR-Cas System (2015); Cell; 163, 1-13).

By “cleavage domain” or “active domain” or “nuclease domain” of a nuclease/endonuclease it is meant the polypeptide sequence or domain within the nuclease which possesses the catalytic activity for polynucleotide cleavage, in particular DNA cleavage. A cleavage domain can be contained in a single polypeptide chain or cleavage activity can result from the association of two (or more) polypeptides. A single nuclease domain may consist of more than one isolated stretch of amino acids within a given polypeptide.

By “recombination” it is meant a process of exchange of genetic information between two polynucleotides. As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the target DNA. In some embodiments, the donor polynucleotide, a portion of the donor polynucleotide, a copy of the donor polynucleotide, or a portion of a copy of the donor polynucleotide integrates into the target genomic region of interest. When HDR requires a «donor» nucleic acid, the genomic region of interest can be defined as the region that is complementary to the «donor» nucleic acid.

By “non-homologous end joining (NHEJ)” it is meant the repair of double-strand breaks in DNA by direct ligation of the break ends to one another without the need for a homologous template (in contrast to homology-directed repair, which requires a homologous sequence to guide repair). NHEJ often results in the insertion or deletion (indel) of one or more nucleotides near the site of the double-strand break.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self 17 hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 8 or 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides).

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

By «silent mutation» it is meant mutations which, when introduced into the genomic region of interest, do not alter the phenotype of the cell and/or organism in which they occur. Silent mutations can occur in non-coding regions (outside of genes or within introns), or they may occur within exons. When silent mutations occur within exons, they either do not result in a change to the amino acid sequence of a protein, or result in the insertion of an alternative amino acid with similar properties to that of the original amino acid. Yet, according to a most preferred embodiment of the invention, «silent mutations consist of mutations which occur within an exon or open-reading frame but that do not result in a change to the amino acid sequence of the protein, or fragment thereof, corresponding to said exon or open-reading frame. Examples of silent mutations include mutations introducing restriction site(s) recognized by one or more endonucleases, but that do not alter the phenotype of the cell and/or organism.

Accordingly, «non-silent mutations» preferably consist of mutations which occur within an exon and that do result in a change to the amino acid sequence of the protein, or fragment thereof, corresponding to said exon. Said change may include deletions, substitutions and insertions of another amino acid sequence. Examples of non-silent mutations include mutations introducing a STOP codon within an open reading frame (ORF).

As used herein a «cell» may encompass the group consisting of eukaryotic and non-eukaryotic cells; which includes eukaryotic cells, and prokaryotic cells selected from bacteria and archaebacterias.

As used herein, an «haploid cell» refers to a cell having a single set of chromosomes;

An «eukaryotic cell» may be selected from the group comprising or consisting of: a yeast, an eukaryotic single-cell organism, a somatic cell, a germ cell, a stem cell, a plant cell, an algal cell, an animal cell, in invertebrate cell, a vertebrate cell, a fish cell, a frog cell, a bird cell, a mammalian cell, a pig cell, a cow cell, a goat cell, a sheep cell, a rodent cell, a rat cell, a mouse cell, a non-human primate cell, and a human cell. Examples of eukaryotic cells which may be considered by the invention include PC9 (lung cancer) cells, BT474 and MCF7 (breast cancer) cells, and DLD-1 and HCT116 (colon cancer), HAP1 (human near-haploid cell line derived from the male chronic myelogenous leukemia (CML) cell line KBM-7) cells.

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is naturally occurring.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9/Csn1 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cas9/Csn1 protein; and a second amino acid sequence other than the Cas9/Csn1 protein). Similarly, “chimeric” in the context of a polynucleotide encoding a chimeric polypeptide includes nucleotide sequences derived from different coding regions (e.g., a first nucleotide sequence encoding a modified or unmodified Cas9/Csn1 protein; and a second nucleotide sequence encoding a polypeptide other than a Cas9/Csn1 protein).

The term “chimeric polypeptide” refers to a polypeptide which is made by the combination (i.e., “fusion”) of two otherwise separated segments of amino sequence, usually through human intervention. A polypeptide that comprises a chimeric amino acid sequence is a chimeric polypeptide. Some chimeric polypeptides can be referred to as “fusion variants.”

“Heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e. a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e. a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., DNA-targeting RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

In particular, the term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol I promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

In Vitro Methods of the Invention

The invention relates to an in vitro method for characterizing one or more genetic variant(s) of a patient.

Hence, according to a first object, the invention thus relates to an in vitro method for characterizing one or more genetic variant(s) of a patient, comprising at least the steps of:

a) bringing into contact a first and a second population of haploid cells with:
a Clustered Regularly Interspaced Short Palindromic Repeats (CRISPR)-CRISPR associated (Cas) (CRISPR-Cas) endonuclease, or an expression system capable of expressing said endonuclease in said first and second population;
a first nucleic acid in conditions suitable for introducing in the first population, after sequence-specific cleavage by the endonuclease, (i) at least one mutation corresponding to the genetic variant(s) of the patient, and (ii) at least one silent, or benign, mutation at the corresponding Protospacer Adjacent Motif (PAM) sequence;
a second nucleic acid in conditions suitable for introducing in the second population, after sequence-specific cleavage by the endonuclease, (i) at least one silent, or benign, mutation at the site of the genetic variant(s), and (ii) at least one silent, or benign, mutation at the corresponding PAM sequence, which is the same as the mutation at the PAM sequence corresponding to the first nucleic acid;

b) culturing said first and second population of haploid cells in a culture medium;

c) determining the occurrence of the genetic variant(s) in the first and second population of haploid cells, thereby characterizing the genetic variant(s) of the patient.

Preferably, the mutation at the corresponding Protospacer Adjacent Motif (PAM) sequence is a silent mutation.

It will be readily understood herein that the efficiency of sequence-specific cleavage by the endonuclease for both conditions should be the same or highly similar in order to compare the genetic variant(s) of both populations of haploid cells, and therefore characterize the genetic variant(s) of the patient.

It will be readily understood herein that CRISPR-Cas systems generally require also the transfection of a guide nucleic acid, in particular of a guide RNA (gRNA) for sequence-specific cleavage. Hence, according to one preferred embodiment, the invention thus relates to an in vitro method for characterizing one or more genetic variant(s) of a patient, comprising at least the steps of:

a) bringing into contact a first and a second population of haploid cells with:
a Clustered Regularly Interspaced Short Palindromic Repeats (CRISPR)-CRISPR associated (Cas) (CRISPR-Cas) endonuclease, or an expression system capable of expressing said endonuclease in said first and second population;
a first nucleic acid in conditions suitable for introducing in the first population, after sequence-specific cleavage by the endonuclease, (i) at least one mutation corresponding to the genetic variant(s) of the patient, and (ii) at least one silent, or benign, mutation at the corresponding Protospacer Adjacent Motif (PAM) sequence;
a second nucleic acid in conditions suitable for introducing in the second population, after sequence-specific cleavage by the endonuclease, (i) at least one silent, or benign, mutation at the site of the genetic variant(s), and (ii) at least one silent, or benign, mutation at the corresponding PAM sequence, which is the same as the mutation at the PAM sequence corresponding to the first nucleic acid;
a guide nucleic acid, such as a guide RNA (gRNA), that hybridizes with a target genomic region of interest, in particular the genomic region of interest including the genetic variant(s) to be characterized;

b) culturing said first and second population of haploid cells in a culture medium;

c) determining the occurrence of the genetic variant(s) in the first and second population of haploid cells, thereby characterizing the genetic variant(s) of the patient.

According to one preferred embodiment of the in vitro method, the first and a second population of haploid cells are brought into contact with a same guide nucleic acid, and more particularly a same guide RNA (gRNA) that hybridizes with the target genomic region of interest.

Advantageously, the said first and second nucleic acid, and the guide nucleic acid (when applicable) are brought simultaneously into contact with the populations of cells.

The expression “mutation at the corresponding Protospacer Adjacent Motif (PAM) sequence” may consist of a mutation on the PAM sequence itself, or around the PAM sequence itself, which may thus consist of a mutation within five (i.e. 1, 2, 3 o, 4 or 5) nucleotides before the PAM sequence in the corresponding sequence (i.e. the sequence targeted by the corresponding guide RNA when applicable).

According to one embodiment, the first and second nucleic acid suitable for introducing, after sequence specific cleavage, a mutation at the site of the genetic variant(s) can be selected from a group consisting of: single-stranded deoxyribonucleotide(s) (ssDNA); double-stranded deoxyribonucleotide(s) (dsDNA); single-stranded ribonucleotide(s) (ssRNA); double-stranded ribonucleotide(s) (dsRNA); single-stranded oligo-deoxyribonucleotide(s) (ssODNA); double-stranded oligo-deoxyribonucleotide(s) (dsODNA); single-stranded oligo-ribonucleotide(s) (ssORNA); double-stranded oligo-ribonucleotide(s) (dsORNA); RNA-DNA duplexes; either in a modified or non-modified form. When the said nucleic acids are in a modified form, they may optionally comprise degenerate sequences and non-standard bases.

For instance, the use of a ssODNA as a donor nucleic acid has been described in: Chen et al. (2011). High-frequency genome editing using ssDNA oligonucleotides with zinc-finger nucleases. Nat Methods. 8(9):753-5.

In a non-limitative manner, said first and second nucleic acid may be in the form of messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers.

According to a preferred embodiment, the first and second nucleic acid are deoxyribonucleic acids, for instance single-stranded oligo-deoxyribonucleotide(s) (ssODNA).

They may be of varying length depending on the nature and length of the genomic region of interest and also for achieving hybridization in the cell and HDR after endonuclease treatment. They may thus comprise or consist of 8, 9, 10, 11, 12, 13, 14, 15, 16, 17, 18, 19, 20, 21, 22, 23, 24, 25, 30, 35, 40, 45, 50, 100, 150 or more nucleotides.

When the first and second nucleic acids are double-stranded nucleic acids, they may either comprise blunt or sticky ends. According to one embodiment, the first and second nucleic acid comprise blunt ends.

According to one embodiment, the first and second nucleic acid are single-stranded oligo-deoxyribonucleotide(s) (ssODNA).

According to one embodiment, the first and second nucleic acid are single-stranded oligo-deoxyribonucleotide(s) (ssODNA) or blunt-ended double-stranded oligo-deoxyribonucleotide(s) (dsODNA).

According to one embodiment of the in vitro method, the first and a second population of haploid cells are brought into contact with a Class II— Clustered Regularly Interspaced Short Palindromic Repeats (CRISPR)-CRISPR associated (Cas) (CRISPR-Cas) endonuclease.

According to one particular embodiment of the in vitro method, the endonuclease belongs to the Type II-CRISPR/Cas endonuclease system, and preferably is a Cas9 or a Cpf1 endonuclease.

In particular, the genetic variant(s) to be characterized is/are classified as Variants of Uncertain Significance (VUS) and/or genetic variant(s) which have not already been classified in databases.

In particular, the genetic variant(s) to be characterized, such as VUS, is/are single nucleotide variants (SNVs), or insertions or deletions (INDELs).

Most preferably, the genetic variant(s) to be characterized by the in vitro method is/are comprised within an essential gene, such as a tumor suppressor gene.

According to one embodiment, the genetic variant(s) to be characterized by the in vitro method is/are comprised within an essential gene, for which loss-of-function results in loss of at least one selected from viability or fitness.

According to one embodiment, the genetic variant(s) to be characterized by the in vitro method is/are comprised within an essential gene, for which loss-of-function results in loss of viability.

According to one embodiment, the genetic variant(s) to be characterized by the in vitro method is/are comprised within an essential gene, for which loss-of-function results in loss of fitness.

According to one alternative embodiment of the in vitro method, the genetic variant(s) is/are not comprised within the gene BRCAL

According to one alternative embodiment of the in vitro method, the genetic variant(s) is/are not comprised within the gene BRCA2.

According to one alternative embodiment of the in vitro method, the genetic variant(s) is/are not comprised within the gene POLE.

According to one alternative embodiment of the in vitro method, the genetic variant(s) is/are not comprised within a gene selected from BRCA1 and BRCA2. According to one alternative embodiment of the in vitro method, the genetic variant(s) is/are not comprised within a gene selected from BRCA1 and POLE.

According to one alternative embodiment of the in vitro method, the genetic variant(s) is/are not comprised within a gene selected from BRCA2 and POLE.

According to one alternative embodiment of the in vitro method, the genetic variant(s) is/are not comprised within a gene selected from BRCA1 and BRCA2 and POLE.

According to one alternative embodiment of the in vitro method, the genetic variant(s) is/are comprised within a gene selected from BRCA1 and BRCA2 and POLE.

In particular, the in vitro method according to the invention can be advantageously applied to a patient having, or which is presumed to have a disorder, for example selected from the group consisting of: a cancer, an autoimmune disease, an inflammatory disease, a neurodegenerative disease.

In particular the invention relates to an in vitro method for characterizing one or more genetic variants, wherein the genetic variant(s) to be characterized is/are comprised within, or associated to, tumor suppressor genes.

The in vitro method according to the invention may thus be advantageously applied to a patient having, or which is presumed to have, a cancer, or a patient having a family history of cancer.

In some particular embodiments, a cancer may include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

Most preferably, the populations of cells which are considered by the methods of the invention consist of haploid eukaryotic cells.

According to one embodiment of the in vitro method, the haploid cells include an inactivated or impaired Non-Homologous End Joining (NHEJ) pathway.

According to one embodiment of the in vitro method, the haploid cells are LIG4 KO or XRCC4 KO cells.

According to one embodiment of the in vitro method, the haploid cells are HAP1 or KBM7 cells.

Advantageously, the in vitro method of the invention does not require any limiting dilution step before being cultured. Hence, according to one embodiment of the in vitro method, the first and second populations of haploid cells are not subjected to limiting dilution before being cultured, thereby avoiding clonal side effects.

According to one embodiment of the in vitro method, the first and second populations of haploid cells are cultured in a suitable culture medium for at least 48 hours, in particular for at least 72 hours, preferably for at least 96 hours, or more.

According to one embodiment, the in vitro method further comprises a step of recovering the first and second population of haploid cells from the culture medium.

According to one embodiment, the in vitro method further comprises a step of recovering genomic DNA, or any nucleic acid sequence derived from said genomic DNA, from the cultured first and second population of haploid cells.

According to one embodiment, the in vitro method further comprises a step of sequencing the genomic DNA, or any nucleic acid sequence derived from said genomic DNA, of the cultured first and second population of haploid cells.

According to one embodiment, the step of determining the occurrence of the genetic variant(s) comprises amplifying the specifically cleaved sequences in the first and second population of haploid cells.

According to one embodiment, the step of determining the occurrence of the genetic variant(s) comprises a step of comparing the level of the genetic variant(s) in the first population of haploid cells to the level of the genetic variant(s) in the second population of haploid cells.

According to one embodiment, the step of determining the occurrence of the genetic variant(s) comprises a determining the frequency of the genetic variant(s) in the first population of haploid cells to the frequency of the genetic variant(s) in the second population of haploid cells.

According to one embodiment, the step of determining the occurrence of the genetic variant(s) in the first and second population of haploid cells consists of determining the occurrence of the mutation corresponding to the genetic variant(s) and to the silent or benign, preferably silent, PAM sequence mutation introduced by the first nucleic acid and comparing it to the occurrence of the silent or benign, preferably silent, mutation at the site of the genetic variant(s) and to the silent PAM sequence mutation introduced by the second nucleic acid.

According to one embodiment, the step of determining the occurrence of the genetic variant(s) in the first and second population of haploid cells consists of determining the mutation frequency corresponding to the genetic variant(s) and to the silent or benign, preferably silent, PAM sequence mutation introduced by the first nucleic acid and the mutation frequency corresponding to the silent mutation at the site of the genetic variant(s) and to the silent or benign, preferably silent, PAM sequence mutation introduced by the second nucleic acid

According to one embodiment, the step of determining the occurrence of the genetic variant(s) in the first and second population of haploid cells consists of determining a function score (FS) with the following formula:

Function score=½*((log 2((fₘᵤₜ*f_{PAMmut})/(fₛᵢₗ*f_{PAMsil})))+(log 2((fₘᵤₜ*f_{PAMsil})/(fₛᵢₗ*f_{PAMmut}))))

with:
fₘᵤₜ for «mutation frequency» (i.e. a VUS of interest in the first population)
f_{PAMmut} for «PAM mutation frequency» (i.e. a silent reference PAM SNV in the first population)
fₛᵢₗ for «silent control frequency» (i.e. a silent control SNV in the second population)
f_{PAMsil} for «PAM mutation frequency» (i.e. a silent reference PAM SNV in the second population).

Advantageously, the determination of the genetic variant(s) in the first and second population of haploid cells, for instance the determination of the corresponding function score for a given variant, allows the characterization of the said variant in the patient.

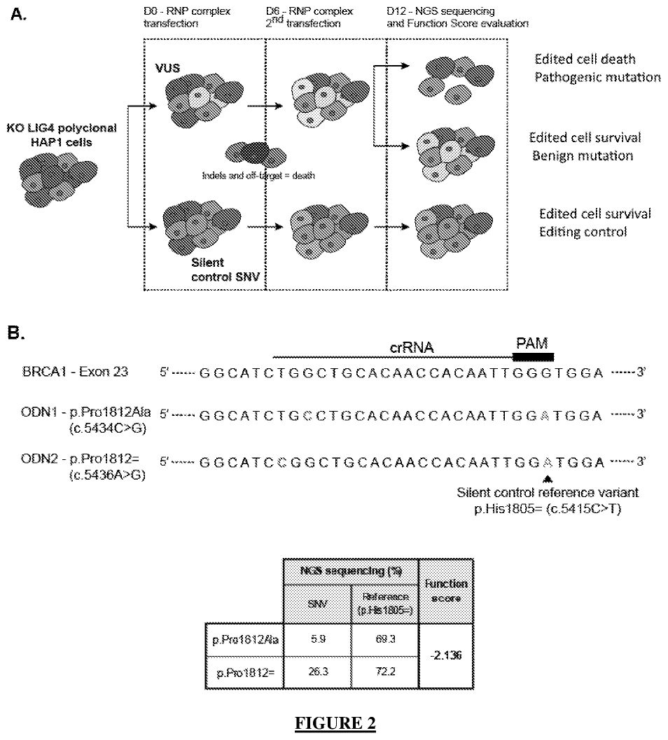

Advantageously, the function score allows its comparison with function scores from other variants. For instance a decreased function score for a given genetic variant is indicative of a pathogenic mutation, when compared to a reference control mutation (see FIG. 2 for an application toward a specific BRCA1 variant centered on Exon 23).

According to one embodiment, the in vitro method further comprises a step of comparing the level of the genetic variant(s) of the first and second population of haploid cells to a reference value. Hence, the in vitro method for characterizing genetic variant(s) in a patient is also suitable as an in vitro method for classifying genetic variant(s) in a patient, and/or for classifying genetic variant(s) in a population of patients.

Therapeutic Applications of the In Vitro Methods

According to a second, alternative, object, the invention relates to a method for preventing or treating a patient bearing a genetic variant, wherein said genetic variant is characterized as pathogenic according to the above-mentioned in vitro method.

The said method thus comprises a step of administering a suitable medication to the patient for which the genetic variant has been characterized.

In particular, the medication may be suitable for preventing the occurrence or re-occurrence, or for reducing the likelihood of occurrence or re-occurence of the disease for which the genetic variant has been characterized as pathogenic.

### EXAMPLES

Material & Methods

HAP1 Cell Culture

Wild-type haploid HAP1 cells were purchased from Horizon Discovery and cultured in Isocove's Modified Dulbecco's Medi (IMDM) containing L-glutamine and 25 mM HEPES (Corning), supplemented with 10% fetal calf serum (Eurobio). Cells were grown at 37° C., under an atmosphere containing 5% CO2 and were passaged before confluence, to prevent reversion to the diploid state. Haploidy of HAP1 cells was confirmed by measure of DNA content via DNA coloration with propidium iodide (PI) dye following vindelov83's method and cytometry analysis before their use.

Genetically Engineered HAP1 Cells

Polyclonal LIG4 knock-out cells were generated with CRISPR-Cas9 technology. A guide RNA (gRNA) was first designed to target the second exon with an AfIII restriction site three nucleotides upstream from the PAM sequence. An Alt-R CRISPR-Cas9 crRNA (IDT DNA) (SEQ ID No 1: 5′-CAATTACACAGTACGTGTCT-3′) and an Alt-R CRISPR-Cas9 tracrRNA with an ATTO550 fluorescent dye (IDT DNA) were complexed at a final concentration of 1 μM with 6 pmol of Alt-R S.p. Hifi Cas9 Nuclease V3 (IDT DNA) in presence of Lipofectamine CRISPRMAX Cas9 Transfection Reagent (Thermo Fisher Scientific). The mixture was incubated for 20 minutes, and reverse transfection was then performed by adding RNA-Cas9 ribonucleoprotein complexes to 1.6×10⁵ cells. Four hours after transfection, cells were sorted by FACS on the basis of ATTO550 fluorescence. Only the 20% of cells with the highest level of fluorescence were retained and used to seed with IMDM supplemented with 1% penicillin-streptomycin (Gibco). The cells were incubated for five days, and then subjected to limiting dilution. About 20 clones were amplified for DNA extraction with Chelex 100 Resin (Biorad). We used 10 μL of these DNA extract for PCR amplification (SEQ ID No 2: forward primer: 5′-CTGGAGAACAGAATTGCAGA-3′; SEQ ID No 3 reverse primer: 5′-TAGCAATCATATTCACGGGC-3′) followed by digestion with the AfIIII restriction enzyme (New England Biolabs) for 1 h at 37° C. The mixture was then incubated for 20 min at 80° C. for enzyme inactivation. The clones were screened by following their migration in a 2% agarose gel on electrophoresis. Clones that had undergone genomic editing and had lost the restriction site where identified by Sanger sequencing on an ABI 3130 Genetic Analyzer (Thermo Fisher Scientific) with the BigDye Terminator v1.1 Cycle sequencing Kit (Thermo Fisher Scientific). Results were visualized and analyzed with Sequencing Analysis 5.3.1 software.

The same technique was used for XRCC4 KO haploid cells using another Alt-R CRISPR-Cas9 crRNA (IDT DNA) (SEQ ID No 4: 5′-ATGGTCATTCAGCATGGACT-3′) and the following primers for amplification (SEQ ID No 5: forward primer 5′-GAGGCCAGTACAGAAAACAT-3′; SEQ ID No 6 reverse primer: 5′-TGGAAAAGTATCCCTGAGGA-3′).

VUS Selection and gRNA Design

The first BRCA1, BRCA2 and POLE variants to be characterized were those found in somatic DNA from patients at the Institut de Cancerologie de l'Ouest (ICO). They were selected for study on the basis of their status as variants of uncertain significance or unclassified variants according to different databases (UMD database, ClinVar, BRCA exchange); well-known mutations were also analyzed as controls. All variants were characterized by Next-Generation Sequencing (NGS) sequencing.

The first stages of NGS library preparation were carried out with the Oncomine BRCA Assay Manual Kit (Thermo Fisher Scientific). Briefly, barcoded libraries were generated from 20 ng of DNA per sample. Two premixed pools of 265 primer pairs for entire BRCA1/2 coding regions and noncoding putative splice boundaries were used to generate the sequencing libraries. Clonal amplification of the libraries was carried out by emulsion PCR using an Ion Chef System (Thermo Fisher Scientific) according to the manufacturer's instructions. The prepared libraries were then sequenced on Ion Torrent S5 Sequencer using Ion 520&530 Kit Chef (Thermo Fisher Scientific). Variants of interest were visualized in Integrative Genomics Viewer (IGV), a high-performance visualization tool for interactive exploration of large integrated genomic datasets on standard desktop computers.

Human Genome Variation Society (HGVS)-approved guidelines (http://www.hgvs.org/mutnomen/) were used for BRCA1 nomenclature. The variants found by NGS were researched in the UMD-BRCA1 database (http://www.umd.be/BRCA1/), the ClinVar database (https://www.ncbi.nlm.nih.gov/clinvar/) and the BRCA Mutation Database (http://www.arup.utah.edu/database/BRCA/Home/BRCA1_landing.php).

A “read” is defined herein as a non-paired sequence having an average length of about 98 bases.

In order to increase the number of characterized VUS, we also selected 10 variants of BRCA1 (p.Ile31Asn, p.Glu149Ala, p.Val191Asp, p.G1n210=, p.Gly462Arg, p.Arg979Cys, p.Gly1201Ser, p.Thr1394Ile, p.Ala1752Pro, p.Gly1770Val) and a POLE variant (p.Arg1826Trp) for which conflicting interpretations had been reported in the databases. Four variants of POLE (p.Ala31Ser, p.Pro286Ser, p.Leu424Val, p.Phe695Ile) were also searched as controls.

Alt-R CRISPR-Cas9 crRNA (IDT DNA) were designed with the Alt-R Custom Cas9 crRNA design tool (IDT DNA) (https://eu.idtdna.com/site/order/designtool/index/CRISPR_CUSTOM).

The PAM sequence had to be adjacent to the mutation to facilitate the editing of KO LIG4 HAP1 cells. The guide RNAs (gRNA) were selected on the basis of the possibility of inserting a silent mutation into the PAM sequence or into the 3 to 5 nucleotides immediately upstream. This increases editing efficiency and will be used as a control in subsequent experiments. For each variant, two Ultramer DNA Oligos (https://eu.idtdna.com/site/order/designtool/index/CRISPR_CUSTOM) (IDT DNA) of about 84nt were designed. The first contained the patient's variant and the second contained a silent mutation, already reported to be benign if possible.

Both contained the silent control mutation mentioned above. All the gRNA and DNA oligonucleotides were designed as described in Table 1 herebelow.

| Genes | Names | RNAg sequences |
|---|---|---|
| BRCA1 | BRAC1-Ile31Asn | TGCTAGTCTGGAGTTGATCA (SEQ ID No 7) |
| BRCA1 | BRAC1-Glu149Ala | TGGCTTCCTGCTAAACAGTA (SEQ ID No 8) |
| BRCA1 | BRAC1-Thr150Ala | TGGCTTCCTGCTAAACAGTA (SEQ ID No 9) |
| BRCA1 | BRAC1-Tyr179Cys | GACGTCTGTCTACATTGAAT (SEQ ID No 10) |
| BRCA1 | BRAC1-Val191Asp | TTCTGAAGATACCGTTAATA (SEQ ID No 11) |
| BRCA1 | BRCA1-Cys197= | TTCTGAAGATACCGTTAATA (SEQ ID No 12) |
| BRCA1 | BRAC1-Gln210= | ATCCAAACTGATTTCATCCC (SEQ ID No 13) |
| BRCA1 | BRAC1-Leu291X | GTTCTCATGCTGTAATGAGC (SEQ ID No 14) |
| BRCA1 | BRAC1-Tyr422X | TGTATTGGACGTTCTAAATG (SEQ ID No 15) |
| BRCA1 | BRAC1-Gly462Arg | TGGGAAAACCTATCGGAAGA (SEQ ID No 16) |
| BRCA1 | BRAC1-Arg979Cys | AAAGTGGTGGTATACGATAT (SEQ ID No 17) |
| BRCA1 | BRCA1-Leu1080= | GAATGCTATGCTTAGATTAG (SEQ ID No 18) |
| BRCA1 | BRAC1-Gly1201Ser | CCCTTTCACCCATACACATT (SEQ ID No 19) |
| BRCA1 | BRAC1-Glu1250Lys | AGACAGACACTCGGTAGCAA (SEQ ID No 20) |
| BRCA1 | BRAC1-Ile1275Val | TAACCAGGTAATATTGGCAA (SEQ ID No 21) |
| BRCA1 | BRCA1-Thr1394Ile | ACACACGCTTTTTACCTGAG (SEQ ID No 22) |
| BRCA1 | BRCA1-Asp1506Asn | ACCACCTATCATCTAATGAT (SEQ ID No 23) |
| BRCA1 | BRAC1-Asp1506Glu | ACCACCTATCATCTAATGAT(SEQ ID No 24) |
| BRCA1 | BRAC1-Glu1586X | GCCAACACGAGCTGACTCTG (SEQ ID No 25) |
| BRCA1 | BRAC1-Gln1604X | TTGGGGAACTTTCAATGCAG (SEQ ID No 26) |
| BRCA1 | BRCA1-Gln1604= | TTGGGGAACTTTCAATGCAG (SEQ ID No 27) |
| BRCA1 | BRCA1-Intron20 | GAAACCAAACACAACCCATC (SEQ ID No 28) |
| BRCA1 | BRAC1-Ala1752Thr | AAAGCGAGCAAGAGAATCCC (SEQ ID No 29) |
| BRCA1 | BRAC1-Ala1752Pro | AAAGCGAGCAAGAGAATCCC (SEQ ID No 30) |
| BRCA1 | BRAC1-Gly1770Val | ACCTGTGGGCATGTTGGTGA (SEQ ID No 31) |
| BRCA1 | BRAC1-Pro1812Ala | CTGGCTGCACAACCACAATT (SEQ ID No 32) |
| BRCA2 | BRCA2-Glu218Lys | CTTACAGCAGTAGTATCATG (SEQ ID No 33) |
| BRCA2 | BRCA2-Asn719Lys | TGATTCTCTGTCATGCCTGC (SEQ ID No 34) |
| BRCA2 | BRCA2-Asp935Asn | ATGGTTTTATATGGAGACAC (SEQ ID No 35) |
| BRCA2 | BRCA2-Lys1l80Arg | TGTCTACCTGACCAATCGAT (SEQ ID No 36) |
| BRCA2 | BRCA2-Ser1882X | AACCTGCCATAATTTTCGTT (SEQ ID No 37) |
| BRCA2 | BRCA2-Val2728Ile | AGATGGGTGGTATGCTGTTA (SEQ ID No 38) |
| BRCA2 | BRCA2-Gln2829Arg | TCAAAGAGCATACCCTATAC (SEQ ID No 39) |
| POLE | POLE-Ala31Ser | AGTTTCGGCACTCAAGCGCC (SEQ ID No 40) |
| POLE | POLE-Pro286Ser | TGTAGGAAATCATCATAATC(SEQ ID No 41) |
| POLE | POLE-Asp301Gly | CACCTGGCCATCGATCATGT (SEQ ID No 42) |
| POLE | POLE-Asn363Asp | CATCATGGTCACCTACAACG (SEQ ID No 43) |
| POLE | POLE-Leu424Val | GGGCAGTCATAATCTCAAGG (SEQ ID No 44) |
| POLE | POLE-Ala456Pro | GAATACGTGGCCAGAGTCTG (SEQ ID No 45) |
| POLE | POLE-Phe695Ile | CCATCGGATCCAGCACCAGC (SEQ ID No 46) |
| POLE | POLE-Argl826Trp | AGCGGTAGAAGTGCATCACC (SEQ ID No 47) |
| Genes | Names | Donor sequences |
| BRCA1 | BRAC1-Ile31Asn-mut | GCAAAATATGTGGTCACACTTTGTGGAGACAGGTTCTTTGTTCAA |
| | | CTCCAGACTAGCAGGGTAGGGGGGGAGAAAAAGAAAATAAATG |
| | | AGGC (SEQ ID No 48) |
| BRCA1 | BRCA1-Ile31Asn-sil | GCAAAATATGTGGTCACACTTTGTGGAGACAGGTTCTTTAATCAA |
| | | CTCCAGACTAGCAGGGTAGGGGGGGAGAAAAAGAAAATAAATG |
| | | AGGC (SEQ ID No 49) |
| BRCA1 | BRAC1-Glu149Ala-mut | CCAAGGTTAGAGAGTTGGACACTGAGACTGGTTGCCTGCTAAAC |
| | | AGTATGATAAAGAACAGTCAAGCAATTGTTGGCCAGTTCTGTGC |
| | | (SEQ ID No 50) |
| BRCA1 | BRAC1-Glu149Ala-sil | CCAAGGTTAGAGAGTTGGACACTGAGACTCGTTTCCTGCTAAAC |
| | | AGTATGATAAAGAACAGTCAAGCAATTGTTGGCCAGTTCTGTGC |
| | | (SEQ ID No 51) |
| BRCA1 | BRAC1-Thr150Ala-mut | CCAAGGTTAGAGAGTTGGACACTGAGACTGGCTTCCTGCTAAAC |
| | | AGTATGATAAAGAACAGTCAAGCAATTGTTGGCCAGTTCTGTGC |
| | | (SEQ ID No 52) |
| BRCA1 | BRAC1-Thr150Ala-sil | CCAAGGTTAGAGAGTTGGACACTGAGACTCGTTTCCTGCTAAAC |
| | | AGTATGATAAAGAACAGTCAAGCAATTGTTGGCCAGTTCTGTGC |
| | | (SEQ ID No 53) |
| BRCA1 | BRAC1-Tyr179Cys-mut | GCAATTATTATTAAATACTTAAAAAACCTGAGACCCTTACCTAAT |
| | | TCAATGCAGACAGACGTCTTTTGAGGTTGTATCCGCTGC (SEQ ID No 54) |
| BRCA1 | BRAC1-Tyr179Cys-sil | GCAATTATTATTAAATACTTAAAAAACCTGAGACCCTTACCTAAT |
| | | TCAATATAGACAGACGTCTTTTGAGGTTGTATCCGCTGC (SEQ ID No 55) |
| BRCA1 | BRAC1-Val191Asp-mut | GGTTCTCTTTGACTCACCTGCAATAAGTTGCTTTATTATCGGTATC |
| | | TTCAGAAGAATCAGATCCTAAAAAATTTCCCCCC (SEQ ID No 56) |
| BRCA1 | BRCA1-Val191Asp-sil | GGTTCTCTTTGACTCACCTGCAATAAGTTGCTTTATTTACGGTATC |
| | | TTCAGAAGAATCAGATCCTAAAAAATTTCCCCCC (SEQ ID No 57) |
| BRCA1 | BRCA1-Cys197=-mut | CCAGCTTCATAGACAAAGGTTCTCTTTGACTCACCTACAATAAGT |
| | | TGCTTTATTAACGGTATCTTCAGAAGAATCAGATCC (SEQ ID No 58) |
| BRCA1 | BRCA1-Cys197=-sil | CCAGCTTCATAGACAAAGGTTCTCTTTGACTCACCTGCAGTAAGT |
| | | TGCTTTATTAACGGTATCTTCAGAAGAATCAGATCC (SEQ ID No 59) |
| BRCA1 | BRCA1-Gln210=-mut | GCCATTACCCTTTTTTGCAGAATCCAAACTGATTTCATCCCTCGTT |
| | | CCCTGAGGGGTGATTTGTAACAATTCTTGATCTCCC (SEQ ID No 60) |
| BRCA1 | BRAC1-Gln210=-sil | GCCATTACCCTTTTTTGCAGAATCCAAACTGATTTCATCCCTCGT |
| | | ACCTTGAGGGGTGATTTGTAACAATTCTTGATCTCCC (SEQ ID No 61) |
| BRCA1 | BRAC1-Leu291X-mut | CTACATTCATTCTGTCTTTAGTGAGTCATAAACTGCTGTTCTCATG |
| | | CTGTAATGAGCTTGCATGAGTATTTGTGCCACATGGCTCC (SEQ |
| | | ID No 62) |
| BRCA1 | BRAC1-Leu291X-sil | CTACATTCATTCTGTCTTTAGTGAGCAATAAACTGCTGTTCTCAT |
| | | GCTGTAATGAGCTTGCATGAGTATTTGTGCCACATGGCTCC (SEQ |
| | | ID No 63) |
| BRCA1 | BRAC1-Tyr422X-mut | CACTGGCCAGTAAGTCTATTTTCTCTGAAGAACCAGACTATTCAT |
| | | CTACTTCATTTAGAACGTCCAATACATCAGCTACTTTGGC (SEQ |
| | | ID No 64) |
| BRCA1 | BRAC1-Tyr422X-sil | CACTGGCCAGTAAGTCTATTTTCTCTGAAGAACCAGAGTATTCAT |
| | | CTACTTCATTTAGAACGTCCAATACATCAGCTACTTTGGC (SEQ |
| | | ID No 65) |
| BRCA1 | BRAC1-Gly462Arg-mut | CAGTTACATGGCTTAAGTTGGGGAGGCTTGCTTTCTTCCGATAGG |
| | | TTTTCCTAAATATTTTGTCTTCAATATTACTCTCTACTG (SEQ ID |
| | | No 66) |
| BRCA1 | BRAC1-Gly462Arg-sil | CAGTTACATGGCTTAAGTTGGGGAGGCTTGCTTTCTTCCGATAGG |
| | | TTTTTCCAAATATTTTGTCTTCAATATTACTCTCTACTG (SEQ ID |
| | | No 67) |
| BRCA1 | BRAC1-Arg979Cys-mut | CTTACATTTAGTTTTAACAAATGACTTGATGGGAAAAAGTGGTGG |
| | | TATACAATATGGATTTTGTAAAAGTCCATGTTTATTTGG (SEQ ID |
| | | No 68) |
| BRCA1 | BRAC1-Arg979Cys-sil | CTTACATTTAGTTTTAACAAATGACTTGATGGGAAAAAGTGGTGG |
| | | TATGCGATATGGATTTTGTAAAAGTCCATGTTTATTTGG (SEQ ID |
| | | No 69) |
| BRCA1 | BRCA1-Leu1080=-mut | GGAAGACTTTGTTTATAGACCTCAGGTTGCAAAACTCCTAATCTA |
| | | AGCATAGCATTCAGTTTTGGCCCTCTGTTTCTACCTAGTTCTGC |
| | | (SEQ ID No 70) |
| BRCA1 | BRCA1-Leu1080=-sil | GGAAGACTTTGTTTATAGACCTCAGGTTGCAAAACACCTAATCTA |
| | | AGCATAGCGTTCAATTTTGGCCCTCTGTTTCTACCTAGTTCTGC |
| | | (SEQ ID No 71) |
| BRCA1 | BRCA1-Gly1201Ser- | GGACTCTAATTTCTTGGCCCCTCTTCGGTAACTCTGAGCGAAATG |
| | mut | TGTATGGGTGAAAGGGCTAGGACTCCTGCTAAGCTCTCC (SEQ ID |
| | | No 72) |
| BRCA1 | BRCA1-Gly1201Ser- | GGACTCTAATTTCTTGGCCCCTCTTCGGTATCCCTGAGCGAAATG |
| | sil | TGTATGGGTGAAAGGGCTAGGACTCCTGCTAAGCTCTCC (SEQ ID |
| | | No 73) |
| BRCA1 | BRCA1- | GCTATTCTTCAATGATAATAAATTCTCCTCTGTGTTCTTAGACAG |
| | Glu1250Lys-mut | ACACTTGGTAGCAACAGTGCTATGCCTAGTAGACTGAGAAGG |
| | | (SEQ ID No 74) |
| BRCA1 | BRCA1- | GCTATTCTTCAATGATAATAAATTCTCCTCTGTGTTCTTAGACAG |
| | Glu1250Lys-sil | ACATTCGGTAGCAACAGTGCTATGCCTAGTAGACTGAGAAGG |
| | | (SEQ ID No 75) |
| BRCA1 | BRAC1-Ile1275Val- | CCTCACTAAGGTGATGTTCCTGAGATGCTTTTGCCAATACTACCT |
| | mut | GGTTACTGCAGTCATTTAAGCTATTCTTCAATGATAATAAATTCT |
| | | CC (SEQ ID No 76) |
| BRCA1 | BRAC1-Ile1275Val- | CCTCACTAAGGTGATGTTCCTGAGATGCTTTTGCCAAGATTACCT |
| | sil | GGTTACTGCAGTCATTTAAGCTATTCTTCAATGATAATAAATTCT |
| | | CC (SEQ ID No 77) |
| BRCA1 | BRCA1-Thr1394Ile- | GCAAAGGACACCACACACACGCATGTGCACACACACACACGCTT |
| | mut | TTTACCTGAATTGTTAAAATGTCACTCTGAGAGGATAGCCC (SEQ |
| | | ID No 78) |
| BRCA1 | BRCA1-Thr1394Ile- | GCAAAGGACACCACACACACGCATGTGCACACACACACACGCTT |
| | sil | TTTACCTGAGTTGTTAAGATGTCACTCTGAGAGGATAGCCC (SEQ |
| | | ID No 79) |
| BRCA1 | BRCA1- | CTCCCAGAGCAACTGTGCATGTACCACCTATTATCTAATGATGGA |
| | Asp1506Asn-mut | CATTTAGAAGGGGATGACCTAGAAAGATAAATGGAAGG (SEQ ID |
| | | No 80) |
| BRCA1 | BRCA1- | CTCCCAGAGCAACTGTGCATGTACCACCTGTCATCTAATGATGGA |
| | Asp1506Asn-sil | CATTTAGAAGGGGATGACCTAGAAAGATAAATGGAAGG (SEQ ID |
| | | No 81) |
| BRCA1 | BRCA1- | CTCCCAGAGCAACTGTGCATGTACCACCTCTCATCTAATGATGGA |
| | Asp1506Glu-mut | CATTTAGAAGGGGATGACCTAGAAAGATAAATGGAAGG (SEQ ID |
| | | No 82) |
| BRCA1 | BRCA1- | CTCCCAGAGCAACTGTGCATGTACCACCTGTCATCTAATGATGGA |
| | Asp1506Glu-sil | CATTTAGAAGGGGATGACCTAGAAAGATAAATGGAAGG (SEQ ID |
| | | No 83) |
| BRCA1 | BRAC1-Glu1586X-mut | GCAGAGGTTGAAGATGGTATGTTGCCAACACGAGCTGACTATGG |
| | | AGCTCTGTCTTCAGAAGGATCAGATTCAGGGTCATCAGAG (SEQ |
| | | ID No 84) |
| BRCA1 | BRAC1-Glu1586X-sil | GCAGAGGTTGAAGATGGTATGTTGCCAACACGAGCTGATTCTGG |
| | | AGCTCTGTCTTCAGAAGGATCAGATTCAGGGTCATCAGAG (SEQ |
| | | ID No 85) |
| BRCA1 | BRAC1-Gln1604X-mut | GCAGCAGCTGGACTCTGGGCAGATTCTGCAACTTTCAATTAGGG |
| | | AACTTTCAATGCAGACGTTGAAGATGGTATGTTGCCAACACGAG |
| | | CTGACTCTGGGGC (SEQ ID No 86) |
| BRCA1 | BRAC1-Gln1604X-sil | GCAGCAGCTGGACTCTGGGCAGATTCTGCAACTTTCAACTGGGG |
| | | AACTTTCAATGCAGACGTTGAAGATGGTATGTTGCCAACACGAG |
| | | CTGACTCTGGGGC (SEQ ID No 87) |
| BRCA1 | BRCA1-Gln1604=-mut | GCAGCAGCTGGACTCTGGGCAGATTCTGCAACTTTCAACTGGGG |
| | | AACTTTCAATGCAGACGTTGAAGATGGTATGTTGCCAACACGAG |
| | | CTGACTCTGGGGC (SEQ ID No 88) |
| BRCA1 | BRCA1-Gln1604=-sil | GCAGCAGCTGGACTCTGGGCAGATTCTGCAACTTTCAATTGTGGA |
| | | ACTTTCAATGCAGACGTTGAAGATGGTATGTTGCCAACACGAGC |
| | | TGACTCTGGGGC (SEQ ID No 89) |
| BRCA1 | BRCA1-Intron20-mut | CCATTGACCACATCTCCTCTGACTTCAAAATCATGCCGAAAGAAA |
| | | CCAAACACAACCCATCACGATAAGAGAAAGAGAAGCTTCCTTCA |
| | | ATGG (SEQ ID No 90) |
| BRCA1 | BRCA1-Intron20-sil | CCATTGACCACATCTCCTCTGACTTCAAAATCATGCTGAAAGAAC |
| | | CCAAACACAACCCATCACGATAAGAGAAAGAGAAGCTTCCTTCA |
| | | ATGG (SEQ ID No 91) |
| BRCA1 | BRAC1-Ala1752Thr- | GAGGGAGGGAGCTTTACCTTTCTGTCTTGGGATTCTCTTGGTCGC |
| | mut | TTTGGACCTTGGTGGTTTCTTCCATTGACCACATCTCC (SEQ ID |
| | | No 92) |
| BRCA1 | BRAC1-Ala1752Thr- | GAGGGAGGGAGCTTTACCTTTCTGTCTTGGGATTCTCTAGCTCGC |
| | sil | TTTGGACCTTGGTGGTTTCTTCCATTGACCACATCTCC (SEQ ID |
| | | No 93) |
| BRCA1 | BRAC1-Ala1752Pro- | GAGGGAGGGAGCTTTACCTTTCTGTCTTGGGATTCTCTTGTTCGC |
| | mut | TTTGGACCTTGGTGGTTTCTTCCATTGACCACATCTCC (SEQ ID |
| | | No 94) |
| BRCA1 | BRAC1-Ala1752Pro- | GAGGGAGGGAGCTTTACCTTTCTGTCTTGGGATTCTCTAGCTCGC |
| | sil | TTTGGACCTTGGTGGTTTCTTCCATTGACCACATCTCC (SEQ ID |
| | | No 95) |
| BRCA1 | BRAC1-Gly1770Val- | GGAACTCTGGGGTTCTCCCAGGCTCTTACCTGTGGGCATGTTGGT |
| | mut | GAACGGCACATAGCAACAGATTTCTAGCCCCCTGAAGATCTGG |
| | | (SEQ ID No 96) |
| BRCA1 | BRAC1-Gly1770Val- | GGAACTCTGGGGTTCTCCCAGGCTCTTACCTGTGGGCATGTTGGT |
| | sil | GAACGGACCATAGCAACAGATTTCTAGCCCCCTGAAGATCTGG |
| | | (SEQ ID No 97) |
| BRCA1 | BRAC1-Pro1812Ala- | GGAAGCCATTGTCCTCTGTCCAGGCATCTGCCTGCACAACCACAA |
| | mut | TTGGATGGACACCCTGGATCCCCAGGAAGGAAAGAGCATTC |
| | | (SEQ ID No 98) |
| BRCA1 | BRAC1-Pro1812Ala- | GGAAGCCATTGTCCTCTGTCCAGGCATCCGGCTGCACAACCACA |
| | sil | ATTGGATGGACACCCTGGATCCCCAGGAAGGAAAGAGCATTC |
| | | (SEQ ID No 99) |
| BRCA2 | BRCA2-Glu218Lys- | CAGTCAGAAATGAAGAAGCATCTAAAACTGTATTTCCTCACGAT |
| | mut | ACTACTGCTGTAAGTAAATATGACATTGATTAGACTGTTG (SEQ |
| | | ID No 100) |
| BRCA2 | BRCA2-Glu218Lys- | CAGTCAGAAATGAAGAAGCATCTGAGACTGTATTTCCTCACGAT |
| | sil | ACTACTGCTGTAAGTAAATATGACATTGATTAGACTGTTG (SEQ |
| | | ID No 101) |
| BRCA2 | BRCA2-Asn719Lys- | GTTATTTATTACCCCAGAAGCTGATTCTCTGTCATGCCTGCAAGA |
| | mut | AGGACAGTGTGAAAAGGATCCAAAAAGCAAAAAAGTTTCAG |
| | | (SEQ ID No 102) |
| BRCA2 | BRCA2-Asn719Lys- | GTTATTTATTACCCCAGAAGCTGATTCTCTGTCATGCCTGCAAGA |
| | sil | AGGACAGTGTGAAAACGATCCAAAAAGCAAAAAAGTTTCAG |
| | | (SEQ ID No 103) |
| BRCA2 | BRCA2-Asp935Asn- | CGAACCCATTTTCAAGAACTCTACCATGGTTTTATATGGAGATAC |
| | mut | AGGTAATAAACAAGCAACCCAAGTGTCAATTAAAAAAGATTTGG |
| | | (SEQ ID No 104) |
| BRCA2 | BRCA2-Asp935Asn- | CGAACCCATTTTCAAGAACTCTACCATGGTTTTATATGGAGATAC |
| | sil | AGGTGACAAACAAGCAACCCAAGTGTCAATTAAAAAAGATTTGG |
| | | (SEQ ID No 105) |
| BRCA2 | BRCA2- | GCTGATCTTCATGTCATAATGAATGCTCCATCGATTGGTCAGGTA |
| | Lys1180Arg-mut | GACAGCAGCAGGCAATTTGAAGGTACAGTTGAAATTAAACGG |
| | | (SEQ ID No 106) |
| BRCA2 | BRCA2- | GCTGATCTTCATGTCATAATGAATGCTCCATCGATTGGTCAGGTA |
| | Lys1180Arg-sil | GACAGCAGCAAACAATTTGAAGGTACAGTTGAAATTAAACGG |
| | | (SEQ ID No 107) |
| BRCA2 | BRCA2-Ser1882X- | CAGTAAAGTAATTAAGGAAAACAACGAGAATAAATAAAAAATTT |
| | mut | GTCAAACGAAAATTATGGCAGGTTGTTACGAGGCATTGG (SEQ |
| | | ID No 108) |
| BRCA2 | BRCA2-Ser1882X- | CAGTAAAGTAATTAAGGAAAACAACGAGAATAAATCCAAAATTT |
| | sil | GTCAAACGAAAATTATGGCAGGTTGTTACGAGGCATTGG (SEQ |
| | | ID No 109) |
| BRCA2 | BRCA2-Val2728Ile- | GATACCCAAAAAGTGGCCATTATTGAACTTACAGATGGGTGGTA |
| | mut | TGCTATTAAAGCCCAGTTAGATCCTCCCCTCTTAGCTGTC (SEQ |
| | | ID No 110) |
| BRCA2 | BRCA2-Val2728Ile- | GATACCCAAAAAGTGGCCATTATTGAACTTACAGATGGGTGGTA |
| | sil | TGCTGTAAAAGCCCAGTTAGATCCTCCCCTCTTAGCTGTC (SEQ |
| | | ID No 111) |
| BRCA2 | BRCA2- | GGAAATGTTGGTTGTGTTGATGTAATTATTCAAAGAGCATACCCT |
| | Gln2829Arg-mut | ATCCGGGTATGATGTATTCTTGAAACTTACCATATATTTC (SEQ |
| | | ID No 112) |
| BRCA2 | BRCA2- | GGAAATGTTGGTTGTGTTGATGTAATTATTCAAAGAGCATATCCT |
| | Gln2829Arg-sil | ATCCAGGTATGATGTATTCTTGAAACTTACCATATATTTC (SEQ |
| | | ID No 113) |
| POLE | POLE-Ala31Ser-mut | CCCTTCTTTCACTCAGGGATGATGGCGCCACTTCCTCAGTTTCGT |
| | | CACTCAAGCGCCTCGAACGGAGTCAGTGGACGGATAAGATGG |
| | | (SEQ ID No 114) |
| POLE | POLE-Ala31Ser-sil | CCCTTCTTTCACTCAGGGATGATGGCGCCACTTCCTCAGTTTCGG |
| | | CCCTCAAGCGCCTCGAACGGAGTCAGTGGACGGATAAGATGG |
| | | (SEQ ID No 115) |
| POLE | POLE-Pro286Ser-mut | GACATTGAGACGACCAAACTGCCCCTCAAGTTTTCTGATGCTGAG |
| | | ACAGATCAGATTATGATGATTTCCTACATGATCGATGGCCAGG |
| | | (SEQ ID No 116) |
| POLE | POLE-Pro286Ser-sil | GACATTGAGACGACCAAACTGCCCCTCAAGTTTCCTGATGCCGA |
| | | GACAGATCAGATTATGATGATTTCCTACATGATCGATGGCCAGG |
| | | (SEQ ID No 117) |
| POLE | POLE-Asp301Gly-mut | GCTGAGACAGACCAGATTATGATGATTTCGTACATGATCGGTGG |
| | | CCAGGTGAGCAGGTGGCTTCTGGGAAGTAAGCTCCTGGG (SEQ |
| | | ID No 118) |
| POLE | POLE-Asp301Gly-sil | GCTGAGACAGACCAGATTATGATGATTTCGTACATGATTGATGG |
| | | CCAGGTGAGCAGGTGGCTTCTGGGAAGTAAGCTCCTGGG (SEQ |
| | | ID No 119) |
| POLE | POLE-Asn363Asp-mut | GGTTTGAACACGTCCAGGAGACCAAACCCACCATCATGGTCACC |
| | | TACGACGGAGACTTTTTTGACTG GTGAGTCTGTGTCTTC (SEQ ID |
| | | No 120) |
| POLE | POLE-Asn363Asp-sil | GGTTTGAACACGTCCAGGAGACCAAACCCACCATCATGGTCACC |
| | | TACAATGGAGACTTTTTTGACTG GTGAGTCTGTGTCTTC (SEQ ID |
| | | No 121) |
| POLE | POLE-Leu424Val-mut | GGTGGGTGAAGAGGGACAGTTACCTTCCTGTGGGCAGTCATAAT |
| | | GTCAAGGCCGCCGCCAAGGCCAAGCTAGGCTATGATCCCG (SEQ |
| | | ID No 122) |
| POLE | POLE-Leu424Val-sil | GGTGGGTGAAGAGGGACAGTTACCTTCCTGTGGGCAGTCATAAT |
| | | CTGAAGGCCGCCGCCAAGGCCAAGCTAGGCTATGATCCCG (SEQ |
| | | ID No 123) |
| POLE | POLE-Ala456Pro-mut | GATGGCCCTGCTCTCTGGCGTTCTCTTCTCAGACTCTGCCCACGT |
| | | ATTCTGTGTCAGATGCTGTCGCCACTTACTACCTGTAC (SEQ ID |
| | | No 124) |
| POLE | POLE-Ala456Pro-sil | GATGGCCCTGCTCTCTGGCGTTCTCTTCTCAGACTCTGGCCACAT |
| | | ATTCTGTGTCAGATGCTGTCGCCACTTACTACCTGTAC (SEQ ID |
| | | No 125) |
| POLE | POLE-Phe695Ile-mut | CAGCCAGTCGCAGCGAATACCATCGGATCCAGCACCAGCTCGAG |
| | | TCAGAGAAGATCCCCCCCTTGTTCCCAGAGGGGCCAGCTCGG |
| | | (SEQ ID No 126) |
| POLE | POLE-Phe695Ile-sil | CAGCCAGTCGCAGCGAATACCATCGGATCCAGCACCAGCTCGAG |
| | | TCAGAGAAGTTTCCCCCCTTGTTCCCAGAGGGGCCAGCTCGG |
| | | (SEQ ID No 127) |
| POLE | POLE-Arg1826Trp-mut | CCCAGTACCACAACATCTATGCAGACAATCAGGTGATGCACTTCT |
| | | ACCGCTGGCTTTGGTCGCCATCCTCTCTGCTTCATGACCC (SEQ ID |
| | | No 128) |
| POLE | POLE-Arg1826Trp-sil | CCCAGTACCACAACATCTATGCAGACAATCAGGTGATGCACTTCT |
| | | ACCGCTGGCTTCGTTCGCCATCCTCTCTGCTTCATGACCC (SEQ ID |
| | | No 129) |

TABLE 2
BRCA1/2 and POLE variants used as silent controls or references
with their functional impact according to different databases.

| | | Amino | | | | | |
|---|---|---|---|---|---|---|---|
| | Nucleotide | acid | | UMD | Clinvar | BRCA | |
| Gene | change | change | Exon | database | database | exchange | Characterized variant |
| BRCA1 | c.93C > T | Ile31= | 3 | ** | ** | ** | Ile31Asn |
| BRCA1 | c.96G > A | Lys32= | 3 | ** | ** | ** | Ile31Asn |
| BRCA1 | c.442-12C > T | | 3 | ** | ** | ** | Glu149Ala, Thr150Ala |
| BRCA1 | c.450C > G | Thr150= | 3 | ** | ** | ** | Glu149Ala, Thr150Ala |
| BRCA1 | c.537C > T | Tyr179= | 8 | ** | ** | ** | Tyr179Cys |
| BRCA1 | c.546G > A | Leu182= | 8 | ** | ** | ** | Tyr179Cys |
| BRCA1 | c.573T > A | Val191= | 8 | ** | ** | ** | Val191Asp |
| BRCA1 | c.579G > A | Lys193= | 8 | ** | * | * | Val191Asp, Cys197Cys |
| BRCA1 | c.591C > T | Tyr196= | 8 | * | * | * | Cys197Cys |
| BRCA1 | c.633A > T | Gly211= | 0 | ** | ** | ** | Gln210Gln |
| BRCA1 | c.636C > G | Thr212= | 0 | * | ** | ** | Gln210Gln |
| BRCA1 | c.840C > A | Ala280= | 1 | ** | ** | ** | Leu291X |
| BRCA1 | c.873A > G | Leu291= | 1 | ** | ** | ** | Leu291X |
| BRCA1 | c.1254G > A | Glu418= | 1 | ** | * | * | Tyr422X |
| BRCA1 | c.1266T > C | Tyr422= | 1 | ** | ** | ** | Tyr422X |
| BRCA1 | c.1386G > A | Gly462= | 1 | ** | * | * | Gly462Arg |
| BRCA1 | c.1404G > A | Lys468= | 1 | ** | * | ** | Gly462Arg |
| BRCA1 | c.2928C > T | Asn976= | 1 | ** | ** | ** | Arg979Cys |
| BRCA1 | c.2937T > C | Arg979= | 1 | ** | ** | ** | Arg979Cys |
| BRCA1 | c.3243T > C | Asn1081= | 1 | ** | * | * | Leu1080= |
| BRCA1 | c.3261G > A | Gly1087= | 1 | ** | ** | ** | Leu1080= |
| BRCA1 | c.3594C > G | Leu1198= | 1 | ** | ** | ** | Gly1201Ser |
| BRCA1 | c.3603T > A | Gly1201= | 1 | ** | ** | ** | Gly1201Ser |
| BRCA1 | c.3738C > T | Thr1246= | 1 | ** | * | * | Glu1250Lys |
| BRCA1 | c.3750G > A | Glu1250= | 1 | ** | * | * | Glu1250Lys |
| BRCA1 | c.3825A > C | Ile1275= | 1 | ** | ** | ** | Ile1275Val |
| BRCA1 | c.3834G > A | Lys1278= | 1 | ** | * | * | Ile1275Val |
| BRCA1 | c.4173G > A | Ile1391= | 2 | ** | ** | ** | Thr1394Ile |
| BRCA1 | c.4179C > A | Thr1393= | 2 | ** | ** | ** | Thr1394Ile |
| BRCA1 | c.4503C > T | Cys1501= | 5 | *** | * | * | Asp1506Asn, Asp1506Glu |
| BRCA1 | c.4518T > C | Asp1506= | 5 | ** | * | * | Asp1506Asn, Asp1506Glu |
| BRCA1 | c.4752C > T | Ala1584= | 5 | ** | * | * | Glu1586X |
| BRCA1 | c.4758G > A | Glu1586= | 5 | ** | ** | ** | Glu1586X |
| BRCA1 | c.4791C > G | Thr1597= | 6 | ** | ** | ** | Gln1604X, Gln1604= |
| BRCA1 | c.4809C > A | Pro1603= | 6 | ** | ** | ** | Gln1604= |
| BRCA1 | c.4812A > G | Gln1604= | 6 | * | * | * | Gln1604X |
| BRCA1 | c.5194-28C > G | | 0 | ** | ** | ** | c.5194-2A>G |
| BRCA1 | c.5194-10T > G | | 0 | ** | ** | ** | c.5194-2A>G |
| BRCA1 | c.5256A > T | Ala1752= | 0 | ** | ** | ** | Ala1752Thr, Ala1752Pro |
| BRCA1 | c.5268G > A | Gln1756= | 0 | ** | * | * | Ala1752Thr, Ala1752Pro |
| BRCA1 | c.5310G > T | Gly1770= | 1 | ** | * | * | Gly1770Val |
| BRCA1 | c.5313C > G | Pro1771= | 1 | ** | ** | ** | Gly1770Val |
| BRCA1 | c.5415C > T | His1805= | 3 | ** | * | ** | Pro1812Ala |
| BRCA1 | c.5436A > G | Pro1812= | 3 | *** | ** | ** | Pro1812Ala |
| BRCA2 | c.654A > G | Glu218= | 8 | ** | ** | ** | Glu218Lys |
| BRCA2 | c.669T > C | His223= | 8 | ** | ** | ** | Glu218Lys |
| BRCA2 | c.2139G > A | Gln713= | 1 | ** | ** | ** | Asn719Lys |
| BRCA2 | c.2157T > C | Asn719= | 1 | ** | ** | ** | Asn719Lys |
| BRCA2 | c.2796C > T | Asp932= | 1 | ** | ** | ** | Asp935Asn |
| BRCA2 | c.2805T > C | Asp935= | 1 | ** | * | * | Asp935Asn |
| BRCA2 | c.3510C > T | Ala1170= | 1 | *** | ** | ** | Lys1180Arg |
| BRCA2 | c.3540G > A | Lys1180= | 1 | ** | ** | ** | Lys1180Arg |
| BRCA2 | c.5646A > C | Ser1882= | 1 | ** | ** | ** | Ser1882X |
| BRCA2 | c.5655C > T | Cys1885= | 1 | ** | ** | ** | Ser1882X |
| BRCA2 | c.8184T > A | Val2728= | 8 | ** | ** | ** | Val2728Ile |
| BRCA2 | c.8187G > A | Lys2729= | 8 | ** | * | * | Val2728Ile |
| BRCA2 | c.8478C > T | Tyr2826= | 9 | ** | ** | ** | Gln2829Arg |
| BRCA2 | c.8484A > C | Ile2828= | 9 | ** | ** | ** | Gln2829Arg |
| POLE | c.93A > C | Ala31= | 2 | * | * | ** | Ala31Ser |
| POLE | c.105G > C | Leu35= | 2 | * | ** | ** | Ala31Ser |
| POLE | c.864T > C | Ala288= | 9 | ** | * | ** | Pro286Ser |
| POLE | c.874C > T | Asp291= | 9 | ** | ** | ** | Pro286Ser |
| POLE | c.891C > G | Ser297= | 9 | ** | ** | ** | Asp301Gly |
| POLE | c.900C > T | Ile300= | 9 | ** | * | ** | Asp301Gly |
| POLE | c.1089C > T | Asn363= | 1 | * | * | ** | Asn363Arg |
| POLE | c.1092G > A | Gly364= | 1 | * | * | ** | Asn363Arg |
| POLE | c.1272C > G | Leu424= | 3 | ** | * | ** | Leu424Val |
| POLE | c.1278G > C | Ala426= | 3 | ** | * | ** | Leu424Val |
| POLE | c.1360-6C > T | | 4 | * | * | ** | Ala456Pro |
| POLE | c.1371G > A | Thr457= | 4 | * | * | ** | Ala456Pro |
| POLE | c.2070G > C | Leu690= | 9 | ** | ** | ** | Phe695Ile |
| POLE | c.2085C > T | Phe695= | 9 | ** | *** | ** | Phe695Ile |
| POLE | c.5448C > T | Asn1815= | 0 | ** | ** | ** | Arg1826Trp |
| POLE | c.5478G > T | Arg1826= | 0 | * | * | ** | Arg1826Trp |

Benign mutations are shown as (*), unreported mutations are shown as (**), variants with conflicting interpretation are shown as (***).

Transfection of LIG4 KO HAP1 Cells

For each variant, two transfections were performed simultaneously, both with the same gRNA but with different DNA oligomers (the VUS to be classified in one transfection 10 or the silent mutation in the other). The same protocol was used, but with 2 nmol of DNA oligonucleotides added before the Lipofectamine CRISPRMAX Cas9 Transfection Reagent. A cell suspension containing 400 000 cells/mL in IMDM supplemented with 10% FBS was then prepared and Alt-R HDR Enhancer (IDT DNA) was added to a final concentration of 2 nM. Reverse transfection was then performed. On day 1 post-transfection, the medium was replaced with fresh Iscove's Modified Dulbecco's Medium (IMDM) supplemented with 10% FBS. On day 4 to 5, depending on the degree of confluence, the cells were released by trypsin treatment and used to seed 6 cm-diameter plates. Two days after plating, a second transfection was performed with the same protocol for both types of transfection, to enrich the cells preparation in edited cells. The cells were then incubated for a further four to five days before DNA extraction.

DNA Extraction and NGS Sequencing

All gDNA were extracted from edited cells with the Maxwell 16 Blood DNA Purification Kit (Promega) and quantified using a Qubit (Thermo Fisher Scientific) and the Quantifluor dsDNA System Kit (Promega). 20 ng of the extracted DNA were then used to generate the NGS library. The libraries were prepared with the Oncomine BRCA Assay Manual Kit or Ion Ampliseq POLE (Thermo Fisher Scientific), allowing amplification of the entire BRCA1 and BRCA2 or POLE coding regions and noncoding putative splice boundaries. Samples were barcoded and the libraries were subjected to clonal amplification by PCR emulsion with an Ion Chef System (Thermo Fisher Scientific). The prepared libraries were then sequenced on an Ion Torrent S5 Sequencer with the Ion 520 and 530 Chef Kit (Thermo Fisher Scientific). Variants of interest were visualized with Integrative Genomics Viewer (IGV) (http://software.broadinstitute.org/software/igv/).

Sequencing Analysis and VUS Function Score Evaluation

Following NGS sequencing, insertions or deletions located around the expected cleavage site, in the eight nucleotides centered on the PAM sequence or the seven nucleotides centered on the VUS, were also counted. Indel frequencies were then calculated by dividing the total amount of indels by the total number of reads. For the evaluation of Single-Nucleotide Variation (SNV) coverage, the ratio of the total numbers of reads for the VUS evaluated and the control SNV was calculated. Finally, function scores for all the variants studied were calculated by comparing the sequence frequencies of all the inserted variants (VUS of interest, silent control SNV and silent reference SNV) and the results contained in the available databases (UMD database, Clinvar, BRCA exchange).

Function Score Determination

The following formula is used:

Function score=½*((log 2((fₘᵤₜ*f_{PAMmut})/(fₛᵢₗ*f_{PAMsil})))+(log 2((fₘᵤₜ*f_{PAMsil})/(fₛᵢₗ*f_{PAMmut}))))

with:
fₘᵤₜ for «mutation frequency» (i.e. a VUS of interest) in the first population
f_{PAMmut} for «PAM mutation frequency» (i.e. a silent reference SNV) in the first population
fₛᵢₗ for «silent control frequency» (i.e. a silent control SNV) in the second population
f_{PAMsil} for «silent PAM mutation frequency» (i.e. a silent reference SNV) in the second population.

Read covers must be similar to the control condition and the tested condition. The same applies to indel frequencies. All the variants measured for a given mutation must be localised on a same read.

Statistics

All statistical analyses were performed with GraphPad Prism analysis software.

Generation of the Polyclonal LIG4 Knock-Out HAP1 Model

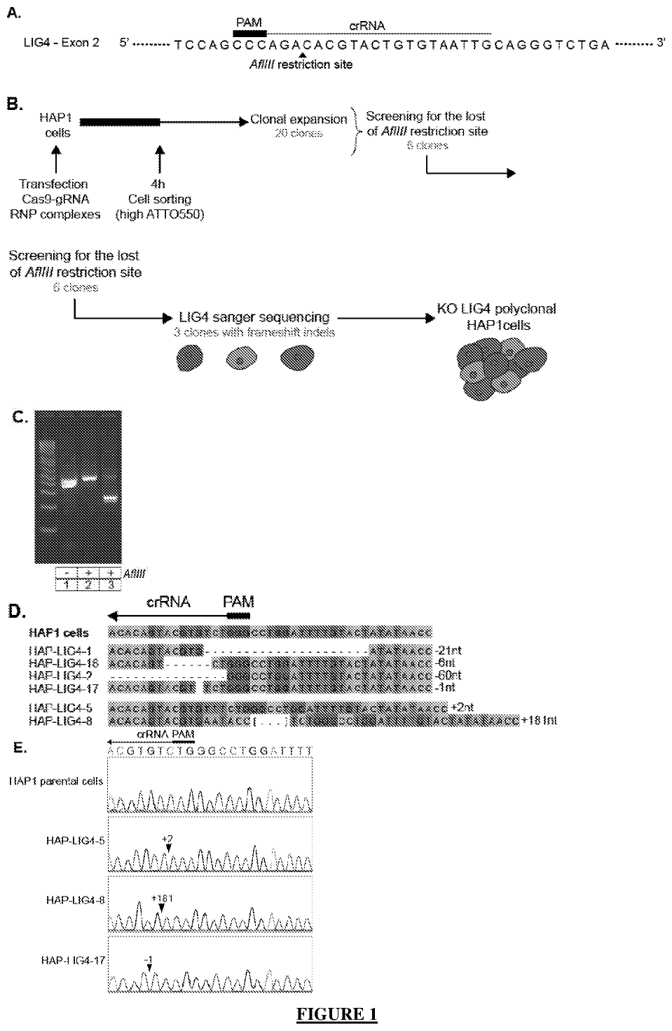

The gRNA targeting this gene was selected according to its proximity to the AflIII restriction site, which is located at the Cas9 double-stranded cleavage site (FIG. 1A). Briefly, cells were transfected and, 4 h later, the cells with the highest level of ATTO550 fluorescence were sorted and subjected to clonal expansion (FIG. 1B). DNA extraction and the PCR amplification of these clones were associated with AflII digestion. The clones that had lost the restriction site were assumed to have undergone editing and were subjected to Sanger sequencing (FIG. 1C). All six of the clones sequenced had been edited, but only three also presented a frameshift (FIGS. 1D and 1E). We pooled 5, 8 and 17 to constitute the polyclonal LIG4 knock-out HAP1 cell line, to prevent side effects and putative off-target effects associated with clonal selection.

### Example

A comparison of editing frequencies between BRCA1/2 variants and silent control SNV can be used for functional classification.

In HAP1 cells, BRCA1 and BRCA2 are essential genes. Genomic editing to create a pathogenic mutation of these genes thus leads to cell death, facilitating the screening of edited cells. Moreover, edited cells with insertions or deletions (hence generating a shift of the reading frame) instead of the mutation of interest also die, due to the essential nature of the gene concerned.

We checked that the absence of a mutation following NGS sequencing was due to the pathogenicity of the mutation rather than a problem linked to genomic editing, by simultaneously performing a second transfection, with the same gRNA, but the insertion of a silent mutation already classified as benign in databases where possible (FIG. 2A). Thus, for each mutation, we designed one gRNA and two oligonucleotides (FIG. 2B), one carrying the mutation of interest and the other carrying a silent control mutation. We also added a second silent mutation, also classified as benign where possible, located in the corresponding PAM motif or 3nt upstream from the PAM motif. This second modification had two purposes. First, its insertion prevented from re-editing blocking gRNA association. This second mutation was present in both sets of conditions, and was therefore also used as reference for comparisons. Following NGS sequencing, mutations frequencies were compared and function score was calculated (FIG. 2B). In our example, the p.Pro1812Ala mutation was pathogenic. We performed two transfections in series, to maximize editing efficiency and mutation frequencies (FIG. 2A).

We then tested our method by using it to characterize 10 variants of BRCA1 and BRCA2 already classified as benign or pathogenic in databases.

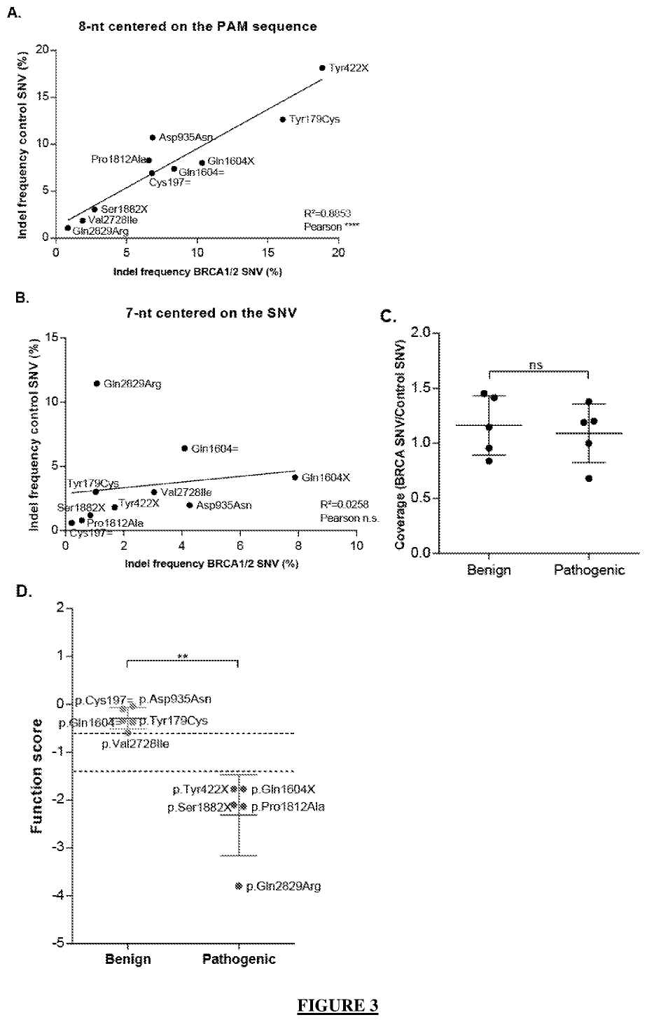

We also evaluated the indel frequency to estimate the editing efficiency in the two conditions compared for each mutation. Indeed, this indel frequency was identical for both conditions when analyzed for the 8nt surrounding the PAM sequence (FIG. 3A) but not for the 7nt surrounding the edited mutation (patient or silent control mutation) (FIG. 3B).

These results confirm that the Cas9 protein cleaves the DNA 3nt upstream from the PAM sequence. Moreover, the observed linear regression made it possible to evaluate genomic editing efficiency and to compare the two conditions with the same gRNA. Following NGS sequencing, the coverage of the mutation of interest (patient or silent control) and the reference control mutation was also checked and shown to be similar in the two conditions (FIG. 3C). Finally, function scores were calculated from sequence frequencies (FIG. 3D). The significant differences observed between benign and pathogenic mutations validated the use of this method to characterize the function of variants of uncertain significance for the BRCA1 and BRCA2 genes.

### Example 2

Functional Characterization of BRCA1/2 Variants of Unknown Significance

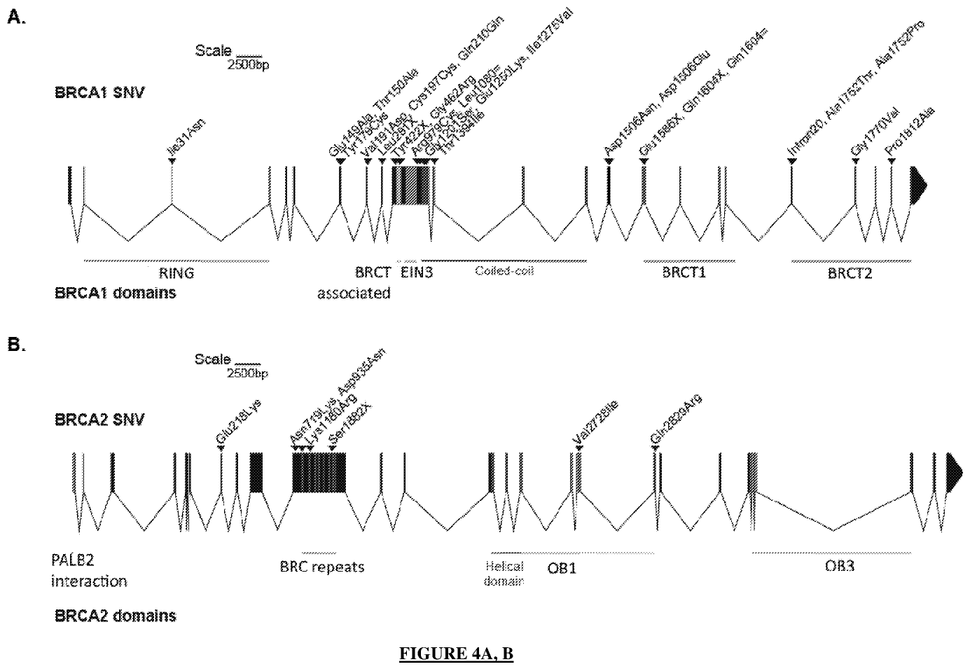

Variants of BRCA1 and BRCA2 were initially selected after characterization in our laboratory, on the basis of an absence of annotation concerning their function. We then also studied other mutations classified as VUS or unreported in the databases (such as UMD database, Clinvar, BRCA exchange . . . ) (Table 1). The 20 BRCA1 and 3 BRCA2 variants affected different domains of the proteins and were distributed along the entire length of these genes (FIGS. 4A and 4B). All reference silent mutations, silent control variants and their classifications in the following databases: UMD database, Clinvar database, BRCA exchange. For each mutation, the coverage (FIG. 4C) and indel frequencies (FIG. 4D) were checked, for the variant of interest and the control. All function scores were then calculated from NGS frequencies (FIG. 4E and Table 1), and the variants were classified according to their impact on cell survival and, consequently, their pathogenicity. Most of the VUS analyzed were neutral (about 65.2%), but six were found to be pathogenic. Another two variants (c.3238T>C and c.5194-2A>G) had intermediate function scores.

We classified 20 of these variants as neutral, six as pathogenic and two as intermediate. These results were consistent with the saturation genome editing study of the RING and BRCT domains of BRCA1 (p.Ile31Asn, p.Ala1752Thr, p.Ala1752Pro, p.Gly1770Val and p.Pro1812A1a) provided in Findlay (“Accurate classification of BRCA1 variants with saturation genome editing”; Nature; 2018).

TABLE 3
Function scores for the evaluated BRCA1/2 variants and comparison with databases annotations.

| | Nucleotide | Amino | | UMD | Clinvar | BRCA | Function | Reclassified |
|---|---|---|---|---|---|---|---|---|
| Gene | change | acid change | xon | database | dtabase | exchange | score | variants |
| BRCA1 | c.92T > A | p.Ile31Asn | 3 | *** | ** | ** | −0.624 | * |
| BRCA1 | c.446A > C | p.Glu149Ala | 3 | *** | *** | *** | 0.361 | * |
| BRCA1 | c.448A > G | p.Thr150Ala | 8 | ** | ** | ** | −0.138 | * |
| BRCA1 | c.536A > G | p.Tyr179Cys | 8 | * | * | * | −0.369 | * |
| BRCA1 | c.572T > A | p.Val191Asp | 8 | *** | *** | ** | −0.229 | * |
| BRCA1 | c.591C > T | p.Cys197= | 8 | * | * | * | −0.096 | * |
| BRCA1 | c.630A > G | p.Gln210= | 10 | *** | *** | *** | −2.055 | ***** |
| BRCA1 | c.872T > G | p.Leu291X | 1 | ** | ** | ** | −2.298 | ***** |
| BRCA1 | c.1266T > G | p.Tyr422X | 1 | ***** | ***** | ***** | −1.771 | ***** |
| BRCA1 | c.1384G > A | p.Gly462Arg | 11 | *** | *** | *** | −0.527 | * |
| BRCA1 | c.2935C > T | p.Arg979Cys | 11 | *** | *** | * | 0.122 | * |
| BRCA1 | c.3238T > C | p.Leu1080= | 11 | *** | * | * | −1.415 | **** |
| BRCA1 | c.3601G > A | p.Gly1201Ser | 11 | *** | * | * | 0.138 | * |
| BRCA1 | c.3748G > A | p.Glu1250Lys | 11 | * | *** | * | 0.117 | * |
| BRCA1 | c.3823A > G | p.Ile1275Val | 11 | * | *** | *** | −0.531 | * |
| BRCA1 | c.4181C > T | p.Thr1394Ile | 12 | *** | *** | *** | 1.430 | * |
| BRCA1 | c.4516G > A | p.Asp1506Asn | 15 | ** | ** | ** | −0.004 | * |
| BRCA1 | c.4518T > G | p.Asp1506Glu | 15 | ** | ** | ** | 0.165 | * |
| BRCA1 | c.4756G > T | p.Glu1586X | 15 | ** | ** | ** | −2.319 | ***** |
| BRCA1 | c.4810C > T | p.Gln1604X | 16 | ***** | * | * | −1.769 | ***** |
| BRCA1 | c.4812A > G | p.Gln1604= | 16 | * | * | * | −0.340 | * |
| BRCA1 | c.5194-2A > G | | 20 | ** | ***** | ***** | −1.123 | **** |
| BRCA1 | c.5254G > A | p.Ala1752Thr | 20 | *** | *** | *** | −2.909 | ***** |
| BRCA1 | c.5254G > C | p.Ala1752Pro | 20 | *** | ***** | *** | −2.235 | ***** |
| BRCA1 | c.5309G > T | p.Gly1770Val | 21 | *** | ***** | ***** | −3.439 | ***** |
| BRCA1 | c.5434C > G | p.Pro1812Ala | 23 | ***** | *** | ***** | −2.136 | ***** |
| BRCA2 | c.652G > A | p.Glu218Lys | 8 | ** | ** | ** | −0.546 | * |
| BRCA2 | c.2157T > G | p.Asn719Lys | 11 | ** | ** | ** | 0.359 | * |
| BRCA2 | c.2803G > A | p.Asp935Asn | 11 | * | * | * | −0.042 | * |
| BRCA2 | c.3539A > G | p.Lys1180Arg | 1 | *** | *** | *** | 0.104 | * |
| BRCA2 | c.5645C > A | p.Ser1882X | 1 | ***** | ***** | ***** | −2.104 | ***** |
| BRCA2 | c.8182G > A | p.Val2728Ile | 8 | * | * | * | −0.598 | * |
| BRCA2 | c.8486A > G | p.Gln2829Arg | 19 | ***** | *** | *** | −3.797 | ***** |

Benign mutations are shown as (*), unreported mutations are shown as (**), mutations with conflicting interpretation are shown as (***), pathogenic mutations are shown as (*****) and intermediate mutations are shown as (****).

Four of the six variants we classified as pathogenic concerned the BRCT domain of BRCA1; the other two were previously unreported nonsense mutations. The results were more surprising for the p.G1n210=variant of BRCA1, which was also found pathogenic. However, this silent mutation may create or strengthen a splice site according to databases. One of the two intermediate variants, c.5194-2A>G, has already been reported to affect splicing and may also be pathogenic. Its classification as functionally intermediate might reflect the existence of a large number of BRCA1 splicing variants. The second intermediate variant, p.Leu1080=, is located in the middle of exon 11 of BRCA1. This synonymous variant has been reported in databases having a likelihood of resulting in a splicing alteration according to bioinformatic analyses. However, our intermediate function score (−1.415) is consistent with the finding of the ESE finder tool, a bioinformatic tool used to identify exonic splicing enhancers, which predicted that this variant might create an SRp40 ESE site (see FIG. 4E). We cannot rule out the possibility that the cut-off used to classify the mutations might be too low, but this value could be refined further after the analysis of more BRCA1/2 variants. Moreover, all variants were compared to silent mutations classified as non-pathogenic or as assumed to be benign. Following NGS sequencing, all these variants were indeed found to be benign.

A three-week period to determine the functional impact of a variant is compatible with clinical management and is one of the main advantages of our protocol (FIG. 2A). Moreover, no clone selection is required, thus avoiding potential off-target effects that could occurred in one cell. This genome editing-based protocol can be used to evaluate exonic, intronic and even splicing variants, by finding a Cas9 PAM sequence (i.e. NGG) close enough to the mutation to edit, to ensure binding of the gRNA. However, increasing numbers of Cas proteins from different species are becoming available, increasing PAM diversity.

We have further tested our protocol with the Cpf1 endonuclease. Similar results were obtained for the variant studied (p.Tyr422X from BRCA1, a pathogenic variant), implying a stable function score.

We have also generalized in another polyclonal cell line, knock-out for the XRCC4 gene, also implicated in the NHEJ pathway. The same function score was obtained for the mutations (p.Ile1275Val, p.Pro1812Ala, p.Tyr422X, p.Gln210=) analyzed in this line, including p.Gln210=variant of BRCA1

The method presented here was proved effective for the characterization of the functional impact of BRCA1 and BRCA2 VUS. More importantly, it can be used to obtain the necessary biological evidence of VUS function required for the prescription of targeted treatment within three weeks, which is compatible with use in clinical application. The patient carrying the genomic abnormality therefore benefits from an analysis of his or her own mutation, with potential consequences for relatives. This is particularly important for extremely rare somatic variants, which resemble orphan diseases.

### Example 3

Extension of the Experimental Process to the Functional Evaluation of POLE Variants

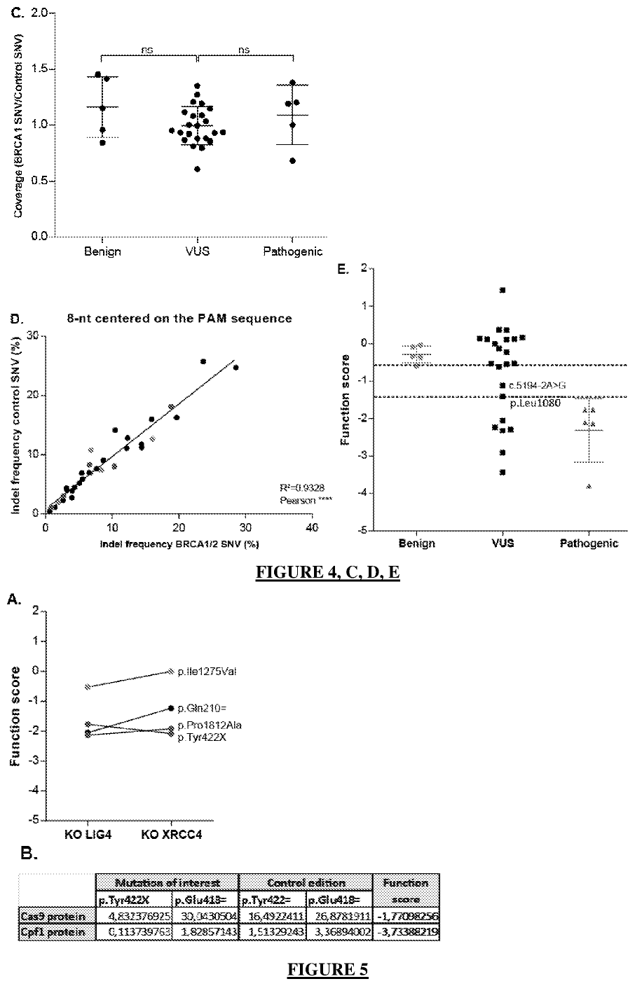

We then extended our protocol to the characterization of VUS from other tumor suppressor genes that were also essential in our model. We chose to study variants of the POLE gene because of potential interest of their functional impact for determining access to immunotherapy. We therefore selected seven POLE mutations from databases, included two classified as benign and two classified as pathogenic (FIG. 5B). The same protocol was followed to evaluate their function scores, and the results obtained were compared with published findings (FIGS. 5A and 5B). The p.Leu424Val variant, which has been classified as pathogenic in databases, was found to have a function score of −0.031.

This supports the extension of the application of our method to the characterization of all essential tumor suppressor genes.

1. An in vitro method for characterizing one or more genetic variant(s) of a patient, comprising at least the steps of:
a) bringing into contact a first and a second population of haploid cells with:
a Clustered Regularly Interspaced Short Palindromic Repeats (CRISPR)-CRISPR associated (Cas) (CRISPR-Cas) endonuclease, or an expression system capable of expressing said endonuclease in said first and second population;
a first nucleic acid in conditions suitable for introducing in the first population, after sequence-specific cleavage by the endonuclease, (i) at least one mutation corresponding to the genetic variant(s) of the patient, and (ii) at least one silent, or benign, mutation at the corresponding Protospacer Adjacent Motif (PAM) sequence;
a second nucleic acid in conditions suitable for introducing in the second population, after sequence-specific cleavage by the endonuclease, (i) at least one silent, or benign, mutation at the site of the genetic variant(s), and (ii) at least one silent, or benign, mutation at the corresponding PAM sequence, which is the same as the mutation at the PAM sequence corresponding to the first nucleic acid;
b) culturing said first and second population of haploid cells in a culture medium;
c) determining the occurrence of the genetic variant(s) in the first and second population of haploid cells, thereby characterizing the genetic variant of the patient.

2. The in vitro method according to claim 1, wherein the mutation at the corresponding Protospacer Adjacent Motif (PAM) sequence is a silent mutation.

3. The in vitro method according to claim 1, wherein
the genetic variant(s) to be characterized is/are comprised within an essential gene, for which loss-of-function results in loss of at least one selected from viability or fitness.

4. The in vitro method according to claim 1, wherein the genetic variant(s) to be characterized is/are Variants of Uncertain Significance (VUS).

5. The in vitro method according to claim 1, wherein the genetic variant(s) to be characterized is/are single nucleotide variants (SNVs), or insertions or deletions (INDELs).

6. The in vitro method according to claim 1, wherein the patient is having, or is presumed to have, a cancer, and/or have a family history of cancer.

7. The in vitro method according to claim 1, wherein the first and a second population of haploid cells are brought into contact with a same guide RNA (gRNA) that hybridizes with a target genomic region of interest.

8. The in vitro method according to claim 1, wherein the first and a second population of haploid cells are brought into contact with a Class II— Clustered Regularly Interspaced Short Palindromic Repeats (CRISPR)-CRISPR associated (Cas) (CRISPR-Cas) endonuclease.

9. The in vitro method according to claim 1, wherein the endonuclease belongs to the Type II-CRISPR/Cas endonuclease system, and preferably is a Cas9 or a Cpf1 endonuclease.

10. The in vitro method according to claim 1, wherein the first and second population of haploid cells are not subjected to limiting dilution before being cultured.

11. The in vitro method according to claim 1, wherein the first and second population of haploid cells are cultured for at least 48 hours, in particular for at least 72 hours, preferably for at least 96 hours.

12. The in vitro method according to claim 1, further comprising a step of recovering the first and second population of haploid cells from the culture medium.

13. The in vitro method according to claim 1, further comprising a step of recovering genomic DNA, or any nucleic acid sequence derived from said genomic DNA, from the cultured first and second population of haploid cells.

14. The in vitro method according to claim 1, further comprising a step of sequencing the genomic DNA, or any nucleic acid sequence derived from said genomic DNA, of the cultured first and second population of haploid cells.

15. The in vitro method according to claim 1, wherein the step of determining the occurrence of the genetic variant(s) comprises a step of comparing the level of the genetic variant(s) in the first population of haploid cells to the level of the genetic variant(s) in the second population of haploid cells.
